(19) United States
(12) Patent Application Publication
(10) Pub. No.: US 2023/0158178 A1
(43) Pub. Date: May 25, 2023

(54) Radionuclide Tracers of 1-Amino-3,4-Difluorocyclopentane-1-Carboxylic Acid, Derivatives, and Uses Thereof

(71) Applicant: Emory University, Atlanta, GA (US)

(72) Inventors: Mark M. Goodman, Atlanta, GA (US); Lanny S. Liebeskind, Atlanta, GA (US); Thomas C. Pickel, Bethel, CT (US)

(21) Appl. No.: 17/908,766

(22) PCT Filed: Mar. 16, 2021
(86) PCT No.: PCT/US2021/022629

§ 371 Date: Sep. 1, 2022

Related U.S. Application Data
(60) Provisional application No. 62/990,198, filed on Mar. 16, 2020.

(51) Int. Cl. A61K 51/04; C07B 59/00

(52) U.S. Cl. CPC A61K 51/0402; C07B 59/001; C07B 2200/05

(57) ABSTRACT

This disclosure relates to 1-amino-3,4-difluorocyclopentane-1-carboxylic acid, esters, derivatives, or salts having positron emitting radionuclides for use as radionuclide tracers. In certain embodiments, 1-amino-3,4-difluorocyclopentane-1-carboxylic acid is isotopically enriched with fluorine 18. In certain embodiments, the radionuclide tracer is amino-3-fluoro-4-[¹⁸F]fluorocyclopentane-1-carboxylic acid. In certain embodiments, the radionuclide tracers are useful to indicate the existence of and/or position of a tumor or cancerous cells in a subject.

### CROSS-REFERENCE TO RELATED APPLICATIONS

This application claims the benefit of U.S. Provisional Application No. 62/990,198 filed Mar. 16, 2020. The entirety of this application is hereby incorporated by reference for all purposes.

### BACKGROUND

Tumors in the brain present a problem to diagnosis and treat due to the involvement of the central nervous system and the challenges of penetrating the blood brain barrier. Gliomas make up a large portion of malignant brain tumors and often result in poor prognosis. Diagnosing can be difficult due to the invasive procedures required to confirm the type of tumor needing to be treated. Techniques, such as MM and CT, provide information on tumor size and general location but the exact boundaries of cancerous cells for the purpose of surgical removal can be difficult to delineate. Thus, there is a need to identify reliable methods of diagnosing and locating brain tumors non-invasively.

Fluciclovine is a fluorine 18 (¹⁸F) labeled synthetic amino acid analog, herein referred to as anti-1-amino-3-[¹⁸F]fluorocyclobutane-1-carboxylic acid or anti-[¹⁸F]FACBC, used as a radioactive diagnostic agent used with PET imaging. Shoup et al. report the synthesis and evaluation of 1-amino-3-[¹⁸F]fluorocyclobutane-1-carboxylic acid to image brain tumors. J Nucl Med. 1999, 40(2):331-8. Oka et al. report the use of anti-1-amino-3-[¹⁸F]fluorocyclobutyl-1-carboxylic acid for the detection of prostate cancer. J Nucl Med. 2007, 48(1):46-55. Savir-Baruch et al. report the evaluation of anti-1-amino-2-[¹⁸F] fluorocyclopentane-1-carboxylic acid (anti-2-[¹⁸F] FACPC) PET-CT in recurrent prostate carcinoma. Mol Imaging Biol. 2011, 13(6):1272-7.

References cited herein are not an admission of prior art.

### SUMMARY

This disclosure relates to 1-amino-3,4-difluorocyclopentane-1-carboxylic acid, esters, derivatives, or salts having positron emitting radionuclides for use as radionuclide tracers. In certain embodiments, 1-amino-3,4-difluorocyclopentane-1-carboxylic acid is isotopically enriched with fluorine 18. In certain embodiments, the radionuclide tracer is 1-amino-3-fluoro-4-[¹⁸F]fluorocyclopentane-1-carboxylic acid. In certain embodiments, the radionuclide tracers are useful to indicate the existence of and/or position of a tumor or cancerous cells in a subject.

In certain embodiments, this disclosure relates to a composition comprising a compound having the following formula (I),

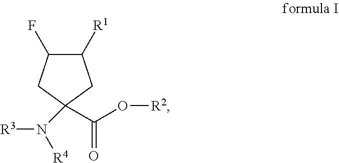

or salt thereof, wherein

R¹ is a radionuclide, alkyl, halogen, hydroxy, amino, ((perfluoroalkyl)sulfonyl))oxy, (arylsulfonyl)oxy, mercapto, alkoxy, or alkylthio, or wherein R¹ is optionally substituted with one or more, the same or different, R¹⁰;

R² is hydrogen, alkyl, formyl, alkanoyl, benzoyl, carbocyclyl, aryl, or heterocyclyl, wherein R² is optionally substituted with one or more, the same or different, R¹⁰;

R³ is hydrogen, alkyl, cyano, hydroxy, amino, formyl, alkanoyl, benzoyl, carboxy, carbamoyl, alkylamino, (alkyl)₂amino, carbocyclyl, aryl, or heterocyclyl, wherein R³ is optionally substituted with one or more, the same or different, R¹⁰;

R⁴ is hydrogen or alkyl; or R³ and R⁴ and attached atoms come together to form a heterocyclyl optionally substituted with one or more, the same or different, R¹⁰;

R¹⁰ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkyl sulfonyl, aryl sulfonyl, alkanoyl, benzoyl, carbocyclyl, aryl, or heterocyclyl, wherein R¹⁰ is optionally substituted with one or more, the same or different, R¹¹; and

R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methyl sulfinyl, ethylsulfinyl, mesyl, ethyl sulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

In certain embodiments, R¹ is a radionuclide.

In certain embodiments, R¹ is ¹⁸F.

In certain embodiments, R¹ is hydroxy optionally substituted with R¹⁰.

In certain embodiments, R¹ is (trifluoromethyl)sulfonyl)oxy.

In certain embodiments, R¹ is a leaving group such as a tin, boron, halogen, hydroxy, or hydroxy substituted with hydroxy protecting group, trifluoromethanesulfonyl, methanesulfonyl, perfluoroalkylsulfonyl, p-toluenesulfonyl, p-bromobenzenesulfonyl, 2-nitrobenzenesulfonyl, or 4-nitrobenzenesulfonyl.

In certain embodiments, R² is ethyl.

In certain embodiments, the compound is 1-amino-3-fluoro-4-[¹⁸F]fluorocyclopentane-1-carboxylic acid, ester, derivative, or salt thereof.

In certain embodiments, the composition comprises a mixture of (1S,3R,4S)-1-amino-3-fluoro-4-[¹⁸F]fluorocyclopentane-1-carboxylic acid and (1R,3S,4R)-1-amino-3-fluoro-4-[¹⁸F]fluorocyclopentane-1-carboxylic acid (anti-cis-3,4-[¹⁸F]-DFACPC) or salts thereof.

In certain embodiments, the composition comprises a mixture of (1S,3S,4R)-1-amino-3-fluoro-4-[¹⁸F]fluorocyclopentane-1-carboxylic acid and (1R,3R,4S)-1-amino-3-fluoro-4-[¹⁸F]fluorocyclopentane-1-carboxylic acid (syn-cis-3,4-[¹⁸F]-DFACPC) of salts thereof.

In certain embodiments, the composition comprises a mixture of (1S,3R,4R)-1-amino-3-fluoro-4-[¹⁸F]fluorocyclopentane-1-carboxylic acid and (1R,3S,4S)-1-amino-3-fluoro-4-[¹⁸F]fluorocyclopentane-1-carboxylic acid (trans-3,4-[¹⁸F]-DFACPC) or salts thereof.

In certain embodiments, the compound is ethyl 1-((tert-butoxycarbonyl)amino)-3-fluoro-4-(((trifluoromethyl)sulfonyl)oxy)cyclopentane-1-carboxylate or salt thereof.

In certain embodiments, the composition comprises a mixture of ethyl (1S,3R,4R)-1-((tert-butoxycarbonyl)amino)-3-fluoro-4-(((trifluoromethyl)sulfonyl)oxy)cyclopentane-1-carboxylate and ethyl (1R,3S,4S)-1-((tert-butoxycarbonyl)amino)-3-fluoro-4-(((trifluoromethyl)sulfonyl) oxy)cyclopentane-1-carboxylate or salts thereof.

In certain embodiments, the composition comprises a mixture of ethyl (1S,3R,4S)-1-((tert-butoxycarbonyl)amino)-3-fluoro-4-(((trifluoromethyl)sulfonyl)oxy)cyclopentane-1-carboxylate and ethyl (1R,3S,4R)-1-((tert-butoxycarbonyl)amino)-3-fluoro-4-(((trifluoromethyl)sulfonyl) oxy)cyclopentane-1-carboxylate or salts thereof.

In certain embodiments, the compound is ethyl 1-(1,3-dioxoisoindolin-2-yl)-3-fluoro-4-(((trifluoromethyl)sulfonyl)oxy)cyclopentane-1-carboxylate or salt thereof.

In certain embodiments, the composition comprises a mixture of ethyl (1S,3S,4S)-1-(1,3-dioxoisoindolin-2-yl)-3-fluoro-4-(((trifluoromethyl)sulfonyl)oxy)cyclopentane-1-carboxylate and ethyl (1R,3R,4R)-1-(1,3-dioxoisoindolin-2-yl)-3-fluoro-4-(((trifluoromethyl)sulfonyl)oxy)cyclo pentane-1-carboxylate or salts thereof.

In certain embodiments, this disclosure relates to pharmaceutical compositions comprising a compound as disclosed herein or salt thereof and a pharmaceutically acceptable excipient.

In certain embodiments, the pharmaceutical composition is in the form of a pH buffered aqueous salt solution between a pH of 4 and 10, 4 and 6, or 6 and 8, or 6 and 10.

In certain embodiments, the pharmaceutical composition is in the form of a saline citrate buffer or phosphate buffer, optionally comprising a saccharide or polysaccharide.

In certain embodiments, this disclosure relates to methods comprising: a) administering a composition comprising compound as disclosed herein containing a radionuclide to a subject; and b) scanning the subject for emissions. In certain embodiments, the method further comprises the step of detecting the emissions and creating an image indicating or highlighting the location of the compound containing radionuclide in the subject.

In certain embodiments, this disclosure relates to kits comprising radionuclide tracers disclosed herein or starting materials to make radionuclide tracers disclosed herein and optionally a substance having an isotopically enriched element for preparing a radionuclide disclosed herein. In certain embodiments, the kit comprises a precursor compound or a compound of formula I.

In certain embodiments, radionuclide tracers are prepared as racemic mixtures. In certain embodiments, the radionuclide tracers have greater than 55%, 60%, 70%, 80%, 90%, or 95% enantiomeric excess or diastereomeric excess.

In certain embodiments, this disclosure relates to methods of preparing compounds disclosed herein comprising mixing starting materials and optionally reagents under conditions such that the products are formed.

### BRIEF DESCRIPTION OF THE SEVERAL VIEWS OF THE DRAWING(S)

FIG. 1 illustrates the structures of racemic mixtures of specific enantiomers of 3,4-difluorocyclopentane1-amino-1-carboxylic acid (DFACPC) isotopically enriched with fluorine 18. The top shows the enantiomers for anti-cis-[¹⁸F]-1-amino-3,4-difluorocyclopentane-1-carboxylic acid or anti-cis-3,4-[¹⁸F] DFACPC or [¹⁸F]3.09. The middle shows the enantiomers for trans-[¹⁸F]-1-amino-3,4-difluorocyclopentane-1-carboxylic acid or trans-3,4-[¹⁸F] DFACPC or [¹⁸F]3.23. The bottom shows the enantiomers for syn-cis-[¹⁸F]-1-amino-3,4-difluorocyclopentane-1-carboxylic acid or syn-cis-3,4-[¹⁸F]-DFACPC or [¹⁸F]3.33.

FIG. 2 illustrates the respective enantiomers of the precursor compounds used to make the radionuclide tracers illustrated in FIG. 1.

FIG. 3 illustrates the structures of several cyclic amino acids.

FIG. 4 shows a table with data on 9 L, U87 ΔEGFR, and DU145 cell uptake of [¹⁸F]3.09, [¹⁸F]3.23, and [¹⁸F]3.33 with and without inhibitors after 30 min of incubation. Data are presented as percent ligand uptake of the initial dose per 0.5 million cells (% ID/5×10⁵ cells).

FIG. 5 shows a table with data on biodistribution of radioactivity in bladders of normal Fischer rats following intravenous administration of syn-cis-3,4-[¹⁸F]-DFACPC, anti-cis-3,4-[¹⁸F] DFACPC, and anti-3-[¹⁸F] FACBC.

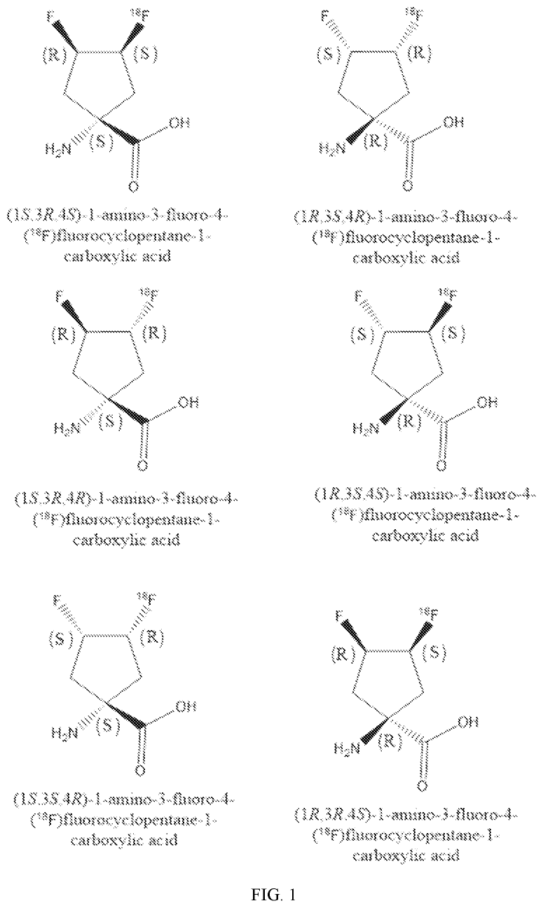

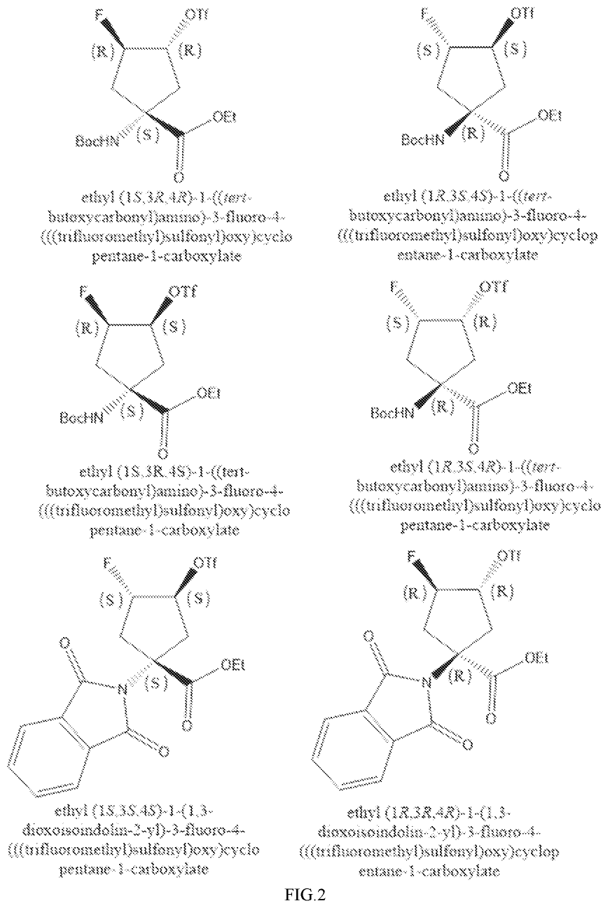

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Prior to describing the various embodiments, the following definitions are provided and should be used unless otherwise indicated.

To the extent that structures provided herein are compounds with tautomers by hydrogen migration, a skilled artisan would understand the formula to cover all tautomeric forms.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

“Positron emission tomography” (PET) refers to an imaging technique that produces an image, e.g., three-dimensional image, by detecting pairs of gamma rays emitted indirectly by a positron-emitting radionuclide tracer. Images of tracer concentration within the area are then constructed by computer analysis. A radioactive tracer is administered to a subject e.g., into blood circulation. Typically, there is a waiting period while tracer becomes concentrated in areas of interest; then the subject is placed in the imaging scanner. As the radionuclide undergoes positron emission decay, it emits a positron, an antiparticle of the electron with opposite charge, until it decelerates to a point where it can interact with an electron, producing a pair of (gamma) photons moving in approximately opposite directions. These are detected in a scanning device. The technique typically utilizes simultaneous or coincident detection of the pair of photons moving in approximately opposite direction (the scanner typically has a built-in slight direction-error tolerance). Photons that do not arrive in pairs (i.e. within a timing-window) are typically ignored. One typically localizes the source of the photons along a straight line of coincidence (also called the line of response, or LOR). This data is used to generate an image.

The term “radionuclide” or “radioactive isotope” refers to molecules of enriched isotopes that exhibit radioactive decay (e.g., emitting positrons). Such isotopes are also referred to in the art as radioisotopes. A radionuclide tracer does not include radioactive primordial nuclides, but does include a naturally occurring isotopes that exhibit radioactive decay with an isotope distribution that is enriched, e.g., is several fold greater than natural abundance. In certain embodiments, is contemplated that the radionuclides are limited to those with a half live of less than 1 hour and those with a half-life of more than 1 hour but less than 24 hours. Radioactive isotopes are named herein using various commonly used combinations of the name or symbol of the element and its mass number (e.g., ¹⁸F, F-18, or fluorine-18).

A “leaving group or atom” is any group or atom that will, under the reaction conditions, cleave from the starting material, thus promoting reaction at a specified site. Suitable non-limiting examples of such groups, unless otherwise specified, include halogen atoms, mesyloxy, p-nitrobenzensulphonyloxy, ((trifluoromethyl)sulfonyl)oxy, and tosyloxy groups.

“Protecting group” has the meaning conventionally associated with it in organic synthesis, e.g., a group that selectively blocks one or more reactive sites in a multifunctional compound such that a chemical reaction can be carried out selectively on another unprotected reactive site and such that the group can readily be removed after the selective reaction is complete. A variety of protecting groups are disclosed, for example, in T. H. Greene and P. G. M. Wuts, Protective Groups in Organic Synthesis, Fourth Edition, John Wiley & Sons, New York (2006), incorporated herein by reference in its entirety. For example, a hydroxy protected form is where at least one of the hydroxy groups present in a compound is protected with a hydroxy protecting group. Likewise, amines and other reactive groups can similarly be protected.

The term “hydroxy protecting group” or “O-protected” as used herein refers to those groups intended to protect an OH group against undesirable reactions during synthetic procedures and which can later be removed to reveal the hydroxyl group. Commonly used hydroxy protecting groups are disclosed in Protective Groups in Organic Synthesis, Greene, T. W.; Wuts, P. G. M., John Wiley & Sons, New York, N.Y., (3rd Edition, 1999). Hydroxy protecting groups include moieties such as allyl, benzyl, methoxymethyl, ethoxyethyl, methyl thiomethyl, benzyloxymethyl, t-butyl, trityl, methoxytrityl, tetrahydropyranyl, 2-napthylmethyl, p-methoxybenzyl, o-nitrobenzyl, 9-phenylxanthyl, silyl groups such as trimethylsilyl, triethylsilyl, triisopropylsilyl, t-butyldimethylsilyl, t-butyldiphenylsilyl, phenyldimethylsilyl, acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, alpha-chlorobutyryl, benzoyl, 4-chlorobenzoyl, 4-bromobenzoyl, 4-nitrobenzoyl, and the like; and sulfonyl groups such as benzenesulfonyl, p-toluenesulfonyl and the like.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isoxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bridge (i.e., —(C═O)alkyl).

“Alkylsulfonyl” refers to an alkyl as defined above attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfinyl” refers to an alkyl as defined above with the indicated number of carbon atoms attached through a sulfinyl bridge (i.e. —S(═O)alkyl).

The terms “halogen” and “halo” refer to fluorine, chlorine, bromine, and iodine.

The term “sulfamoyl” refers to the amide of sulfonic acid (i.e., —S(═O)₂NRR′).

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom or replacing an amino group with a hydroxy group. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze, hereby incorporated by reference.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol or acetamide, formamide, methanesulfonate, and benzamide derivatives of an amine functional group in the active compound and the like.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxy, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

### Tracer Radionuclides

Radionuclides are isotopically labeled forms of compounds disclosed herein including isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁵O, ¹⁷O, ³¹P, ³²P, ³⁵S, ¹⁸F, ³⁶Cl, ¹²⁵I and ¹³¹I. It will be understood that compounds of the disclosure can be labeled with an isotope of any atom or combination of atoms in the structure. While [¹⁸F] has been emphasized herein as being particularly useful for PET, SPECT and tracer analysis, other uses are contemplated including those flowing from physiological or pharmacological properties of stable isotope homologs and will be apparent to those skilled in the art.

Such isotopically labeled compounds are useful in metabolic studies, reaction kinetic studies, detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Isotopically labeled compounds of this disclosure and prodrugs thereof can generally be prepared by carrying out the procedures disclosed in the schemes or in the examples and preparations described herein by substituting a readily available isotopically labeled reagent for a non-isotopically labeled reagent.

In certain embodiments, compounds disclosed herein are substituted with ¹⁸F, Flurone-18. Radiofluorination reactions are typically nucleophilic substitutions. Aromatic nucleophilic substitutions with fluoride usually require activated aromatic rings, bearing both a good leaving group (e.g. a halogen, a nitro- or a trimethylammonium group) and a strong electron-withdrawing substituent (e.g. a nitro-, cyano- or acyl group) preferably placed para to the leaving group, whereas aliphatic nucleophilic substitutions typically utilize leaving group (usually a halogen or a sulfonic acid derivative such as mesylate, tosylate, or triflate).

One can produced [¹⁸F] fluoride by irradiation of water (containing H₂¹⁸O) with protons resulting in the reaction ¹⁸O(p,n)¹⁸F. For production efficiency and radiochemical purity, it is desirable to use water that is as highly enriched as possible. The [¹⁸F] isotope is then separated from water and processed for production of a radiopharmaceutical agent. Typically, fluoride recovery is based on ion exchange resins. The recovery is carried out in two steps (extraction and elution): first the anions (not only fluoride) are separated from the enriched [¹⁸O] water and trapped on a resin and then, said anions, including [¹⁸F]fluoride, are eluted into a mixture containing water, organic solvents, a base, also called activating agent or phase transfer agent or phase transfer catalyst, such as the complex potassium carbonate-Kryptofix 222™ (K₂CO₃—K₂₂₂) or a tetrabutylammonium salt. Kryptofix 222™ is a cyclic crown ether, which binds the potassium ion, preventing the formation of ¹⁸F-KF. Thus, potassium acts as the counter ion of ¹⁸F⁻ to enhance its reactivity but does not interfere with the synthesis. Typical labeling methods use low water content solutions. An evaporation step may follow the recovery of the [¹⁸F]fluoride, e.g., azeotropic evaporation of acetonitrile or other low boiling temperature organic solvent.

Alternatively, the extraction process is performed by passing the [¹⁸F] aqueous solution on a solid support as reported in U.S. Pat. No. 8,641,903. This solid support is typically loaded with a trapping agent, e.g., compound comprising a quaternary amine that is adsorbed on the solid support and allows the [¹⁸F] activity to be trapped because of its positive charge. The solid support is then flushed with a gas or a neutral solvent to remove or push out most of the residual water. The [¹⁸F] is at last eluted in an organic solvent or in a mixture of organic solvents and is usable for the labelling of precursor compounds.

In certain embodiments, compounds disclosed herein are substituted with ¹³N, Nitrogen-13. One can produce [¹³N]NH₃, ammonium with nitrogen-13 (¹³N) in an cyclotron by using the ¹⁶O(p,alpha)¹³N nuclear reaction. One can irradiate EtOH in water at 18 MeV protons (22 mA beam current), pressure in the range 5-10 bar into the target during bombardment to reach integrated currents (0.1-1 mAh). See Da Silva et al. Efficient Enzymatic Preparation of 13N-Labelled Amino Acids: Towards Multipurpose Synthetic Systems, Chem. Eur. J. 2016, 22, 13619.

The compounds described herein could also be labeled by radionuclide bromine or iodine through traditional labeling procedures such as tributyltin derivatives. (See, for example, Plisson et al., Synthesis and in vivo evaluation of fluorine-18 and iodine-123 labeled 2-beta-carbo-(2-fluoroethoxy)-3beta-(4′-((Z)-2 iodoethenyl)phenyl)nortropane as a candidate serotonin transporter imaging agent. J Med Chem, 2007, 50(19):4553-60; Plisson et al, Synthesis, radiosynthesis, and biological evaluation of carbon-11 and iodine-123 labeled 2beta-carbomethoxy-3-beta-[4′-((Z)-2-haloethenyl)phenyl]tropanes. J Med Chem, 2004, 47(5):1122-35; Li et al, Synthesis of structurally identical fluorine-18 and iodine isotope labeling compounds for comparative imaging. Bioconjug Chem, 2003, 14(2):287-94; Goodman et al., Synthesis and characterization of iodine-123 labeled 2-beta-carbomethoxy-3-beta-(4′-((Z)-2-iodoethenyl)phenyl)nortropane. J Med Chem, 2003, 46(6):925-35; Maziere et al, ⁷⁶Br-beta-CBT, a PET tracer for investigating dopamine neuronal uptake. Nucl Med Biol, 1995, 22(8):993-7).

In certain embodiments, compounds disclosed herein may contain ¹¹C, carbon-11. Methods of preparing ¹¹C intermediates are provided in the art. Example of such methods are disclosed in, for example: Jewett et al. (1992) A Simple Synthesis of [¹¹C]Methyl Triflate Appl. Radiat. Isot. 43, 1383-1385; Crouzel et al. (1987) Recommendations for a practical production of [¹¹C]methyl iodide Appl. Radiat. Isot. Int. J. Appl. Instrum. Part A 38, 601-603; Jewett et al. (1991) Captive Solvent Methods for Fast Simple Carbon-11 Radioalkylations. In: New Trends in Radiopharmaceutical Synthesis, Quality Assurance and Regulatory Control (Edited by Emran, A. M.) pp. 387-391. Plenum Press, New York; Marazano, et al. (1977) Synthesis of methyl iodide-¹¹C and formaldehyde-¹¹C Appl. Radiat. Isot. 28, 49-52; Watkins et al. (1988) A Captive Solvent Method for Rapid N-[¹¹C]Methylation of Secondary Amides Application to the Benzodiazepine, 4′-Chlorodiazepam (RO5-4864) Appl. Radiat. Isot. 39, 441-444; and Wilson et al., (1996) In vivo evaluation of [¹¹C] and [¹⁵F]-labeled cocaine analogues as potential dopamine transporter ligands for positron emission tomography. Nucl. Med. Biol. 23, 141-146.

Other halogen isotopes can serve for PET or SPECT imaging, or for conventional tracer labeling. These include ⁷⁵Br, ⁷⁶Br, ⁷⁷Br and ⁸²Br as having usable half-lives and emission characteristics. In general, the chemical means exist to substitute any halogen moiety for the described isotopes. Astatine can be substituted for other halogen isotopes, [²¹⁰At] emits alpha particles with a half-life of 8.3 h. At-substituted compounds are therefore useful for tumor therapy where binding is sufficiently tumor-specific.

### Methods of Use

After a compound disclosed herein comprising a radionuclide is administered to a subject, the subject is then imaged. The radionuclide can be administered at any suitable dose. The subject can be imaged using any suitable imaging apparatus, for example an apparatus capable of gathering a magnetic resonance image (MM), a positron emission tomogram (PET scan) or a computer tomogram (CT scan).

In certain embodiments, the compounds disclosed herein are labeled with a radionuclide suitable for imaging with gamma, PET or SPECT imaging technology, preferably an isotope suitable for PET imaging. In other embodiments, the compounds described herein are labeled with ¹¹C or ¹³C, for example by incorporating into the carbons of the compounds, for Mill or MRS imaging. In other embodiments, the compounds described herein are labeled with a dye, for example, a near-infrared dye, suitable for optical imaging. Exemplary compositions described here can be used to image, detect, and/or predict cancer, in particular the spread of cancer, within an organism.

Instruments for detecting and monitoring by radionuclide imaging the location of a tracer in the body of a subject include positron emission tomography (PET) and single photon emission computed tomography (SPECT) scanners. These may be combined with other methods such as computerized tomography (CT) scans and MM. A CT scan combines a series of X-ray images taken from different angles and uses computer processing to create cross-sectional images, or slices, of the bones, blood vessels and soft tissues inside your body. These scans or associated data can be used to create computerized images that take place in tissue. A scanner records data that a computer constructs two- or three-dimensional images. In a typical method, radioactive compound is injected into the subject, e.g., a vein, and a scanner is used to make detailed images of areas inside the body where the radioactive material is taken up by the cells, tissue, fluids, or organs. For example, when imaging for lymphoma, the scans can show the uptake of the radionuclides in the lymph nodes, the groin, both axilla (armpit), and neck.

In certain embodiments, the disclosure relates to imaging methods comprising a) administering a compound comprising a radionuclide or positron-emitting radionuclide disclosed herein to a subject; and b) scanning the subject for the emission, positron-emissions or other gamma-emissions. The methods typically further comprise the steps of detecting the emissions and creating an image of an area of the subject indicating or highlighting the location of the compound containing radionuclide in the subject. In certain embodiments, the area of the subject is the brain, lymph nodes, groin, axilla, neck, lungs, liver, kidney, pancreas, stomach, balder, intestines, circulatory system, breast, prostate, or gallbladder.

The compound may be administered by any suitable technique known in the art, such as direct injection. Injection may be intravenous (IV). Administration may be general or local to the site of interest, such as into a tumor. The compound may be used in conjunction with another probe, for example a fluorescent probe capable of visualizing a particular tissue or a tumor. The two (or more) probes may be administered together, separately or sequentially. The imaging compound of the present disclosure may be used to diagnose, assess or monitor the progression or treatment of a disease or condition.

Radionuclides of the present disclosure may be used to investigate the effects of a test compound. For example, compounds with a radionuclide may be administered together with a test compound, to evaluate the effect of the test compound be assayed in real time in vivo using a method in accordance with the present disclosure.

The compounds of the disclosure are useful as tracer compounds for tumor imaging techniques, including PET and SPECT imaging. Particularly useful as an imaging agent are those compounds labeled with F-18 since F-18 has a half-life of 110 minutes, which allows sufficient time for incorporation into a radio-labeled tracer, for purification and for administration into a human, mammal, or animal subject. In certain embodiments, dosing or activity at the time of injection is in the range from 10 MBq to 1000 MBq or 50 MBq to 800 MBq or 200 MBq to 400 MB q.

SPECT imaging employs isotope tracers that emit high energy photons (γ-emitters). The range of useful isotopes is greater than for PET, but SPECT provides lower three-dimensional resolution. Nevertheless, SPECT is widely used to obtain clinically significant information about analog binding, localization and clearance rates. A useful isotope for SPECT imaging is [¹²³I], a γ-emitter with a 13.3 hour half-life.

Accordingly, the compounds of the disclosure can be rapidly and efficiently labeled with [¹²³I] for use in SPECT analysis as an alternative to PET imaging. Furthermore, because of the fact that the same compound can be labeled with either isotope, it is possible to compare the results obtained by PET and SPECT using the same tracer.

In certain embodiments, the disclosure provides methods for tumor imaging using PET and SPECT. The methods entail administering to a subject (which can be human or animal, for experimental and/or diagnostic purposes) an image-generating amount of a compound of the disclosure, labeled with the appropriate isotope and then measuring the distribution of the compound by PET if [¹⁸F] or other positron emitter is employed, or SPECT if [¹²³I] or other gamma emitter is employed. An image-generating amount is that amount which is at least able to provide an image in a PET or SPECT scanner considering the detection sensitivity and noise level of the scanner, the age of the isotope, the body size of the subject and route of administration.

Methods of use of the imaging agents provided herein include, but are not limited to: methods of imaging tissue; methods of imaging precancerous tissue, cancer, and tumors; methods of treating precancerous tissue, cancer, and tumors; methods of diagnosing precancerous tissue, cancer, and tumors; methods of monitoring the progress of precancerous tissue, cancer, and tumors; methods of imaging abnormal tissue, and the like. The methods can be used to detect, study, monitor, evaluate, and/or screen, biological events in vivo or in vitro.

In diagnosing and/or monitoring the presence of cancerous cells, precancerous cells, and tumors in a subject, labeled compounds are administered to the subject in an amount effective to result in uptake of the tracer compound into the cells or binding to the labeled compound. After administration of the tracer compounds, cells that take up or bind with the tracer compound are detected using PET or SPECT imaging. Embodiments of the present disclosure can non-invasively image tissue throughout an animal or patient.

It should be noted that the amount effective to result in uptake of the tracer compound into the cells or tissue of interest will depend upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

Preferred imaging methods provided by the present disclosure include the use of the radionuclide containing compounds of the present disclosure and/or salts thereof that are capable of generating at least a 2:1 target to background ratio of radiation intensity, or more preferably about a 5:1, about a 10:1 or about a 15:1 ratio of radiation intensity between target and background. In certain preferred methods, the radiation intensity of the target tissue is more intense than that of the background. In other embodiments, the present disclosure provides methods where the radiation intensity of the target tissue is less intense than that of the background. Generally, any difference in radiation intensity between the target tissue and the background that is sufficient to allow for identification and visualization of the target tissue is sufficient for use in the methods of the present disclosure.

In preferred methods of the present disclosure, the compounds of the present disclosure are excreted from tissues of the body quickly to prevent prolonged exposure to the radiation of the radiolabeled compound administered to the patient. In a particular embodiment, the radionuclide tracer provided herein can be used on an outpatient basis. Typically, compounds of the present disclosure are eliminated from the body in less than about 24 hours. More preferably, compounds of the present disclosure are eliminated from the body in less than about 16 hours, 12 hours, 8 hours, 6 hours, 4 hours, 2 hours, 90 minutes, or 60 minutes.

Preferred imaging agents are stable in vivo such that substantially all, e.g., more than about 50%, 60%, 70%, 80%, or more preferably 90% of the injected compound is not metabolized by the body prior to excretion.

Typical subjects to which compounds of the present disclosure may be administered will be mammals, particularly primates, especially humans. For veterinary applications, a wide variety of subjects will be suitable, e.g. livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine or tissue samples, or blood urine or tissue samples of the animals mentioned for veterinary applications.

Images can be generated by virtue of differences in the spatial distribution of the imaging agents that accumulate at a site. The spatial distribution may be measured using any imaging apparatus suitable for the particular label, for example, a gamma camera, a PET apparatus, a SPECT apparatus, MRS, MRI or optical imaging apparatus, and the like. The extent of accumulation of the imaging agent may be quantified using known methods for quantifying radioactive emissions. A particularly useful imaging approach employs more than one imaging agent to perform simultaneous studies. Alternatively, the imaging method may be carried out a plurality of times with increasing administered dose of the pharmaceutically acceptable imaging composition of the present disclosure to perform successive studies using the split-dose image subtraction method, as are known to those of skill in the art.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

In one embodiment, a method of imaging malignant cell is provided that includes administering a compound disclosed herein to a subject. The malignant cell can be a tumor cell. In certain embodiments, the compound can be provided to a host before treatment of a tumor. In a separate embodiment, the compound is provided to a patient that has been treated for cancer to reduce the likelihood of recurrence, or reduce mortality associated with a particular tumor. In another embodiment, the compound is administered to a host at high risk of suffering from a proliferative disease. Such high risk can be based, for example, on family history or on a history of exposure to known or presumed carcinogens.

Subjects, including humans suffering from, or at risk for, a proliferative disorder can be treated by administering an effective amount of the imaging agent or a pharmaceutically acceptable prodrug or salt thereof in the presence of a pharmaceutically acceptable carrier or diluent. The imaging agent or composition comprising the imaging agent can be administered by any appropriate route, for example, orally, parenterally, intravenously, intradermally, subcutaneously, or topically, in liquid or solid form.

In certain embodiments, this disclosure relates to methods comprising: a) administering a compound disclosed herein containing a radionuclide to a subject and b) scanning the subject for emissions so that an image can be created or the location of the radionuclide can be identified or tracked. In certain embodiments, the image is used to detect malignancies. In certain embodiments, the disclosure contemplates surgical removal of tissue of the subject based on or in the location of the radionuclide in the subject.

In certain embodiments, the subject had an abnormality identified by a prior test and are seeking a diagnosis, or the subject had an existing diagnosis of cancer and are having further monitoring. In certain embodiments, the subject is asymptomatic and used for an early cancer screen or detection.

In certain embodiments, this disclosure relates to method for detecting cancerous or proliferating cells in tissue in a mammal comprising administering to said mammal a compound disclosed herein, or a pharmaceutically acceptable salt thereof, wherein said compound comprises said positron emitting radionuclide, and detecting any of the compound retained in said tissue by positron emission tomography (PET) and/or single photon emission computed tomography (SPECT).

In certain embodiments, the disclosure contemplates methods of treating cancer or other proliferative disorder comprising administering an effective amount of an anticancer agent to a subject in need thereof, optionally in combination with a compound or derivative of 1-amino-3,4-difluorocyclopentane-1-carboxylic acid for use in imaging.

### Kits

In certain embodiments, this disclosure relates to kits comprising radionuclide compounds and/or precursor compounds disclosed herein and instructions for use. In certain embodiments, the instructions provide for the activity at the end of synthesis. In certain embodiments, the instructions provide for the half-life or the radionuclide. In certain embodiments, the instructions provide that injection should be used within limited time from the time of the end of synthesis. In certain embodiments, the container is a sealed container such as a septum capped vial.

In certain embodiments, this disclosure relates to kits comprising precursor compounds, starting materials to make radionuclide tracers disclosed herein, and/or a substance having a mixture for preparing a radionuclide in a cyclotron. In certain embodiments, the kit comprises precursor compounds disclosed herein. In certain embodiments, the kit comprises a container having water, H₂¹⁸O and/or ethanol in water solution. In certain embodiments, the container is sealed from the atmosphere. In certain embodiments, the kits comprise a solid support or filter. In certain embodiments, the filter may be used to purify a radionuclide tracer disclosed herein.

In certain embodiments, the kit comprises a compound disclosed herein. In certain embodiments, the kit comprising a compound disclosed herein with a hydroxy group and the kit further comprises a reagent for generating a leaving group. In certain embodiments, the reagent for generating a leaving group is trifluoromethanesulfonic anhydride, methanesulfonyl chloride, trifluoromethanesulfonyl chloride, p-toluenesulfonyl chloride, p-bromobenzenesulfonyl chloride, 2-nitrobenzenesulfonyl chloride, or 4-nitrobenzenesulfonyl chloride.

It is contemplated that precursor compounds are labeled with radionuclides using methods reported herein to provide the tracers. These tracers may be prepared at the location of the subject near the time the subject is exposed to an imaging device. Thus, in certain embodiments, the disclosure contemplates kits comprising compounds or precursor compounds (e.g., compounds that react with recently generated ¹⁸F), disclosed herein, e.g., compounds disclosed herein comprising substituted with triflate, tosylate and mesylate groups and a solid support.

In certain embodiments, the disclosure contemplates a kit comprising compounds disclosed herein or precursor compounds comprising substituted with halogen, hydroxyl, thiol, —O-p-toluenesulfonyl, —O-p-bromobenzenesulfonyl, —O-(2- or 4)-nitrobenzene sulfonyl, —O— methanesulfonyl, —O-trifluoromethanesulfonyl, —O-5(dimethylamino)naphthalene-1-sulfonyl, —S-p-toluenesulfonyk-S-p-bromobenzenesulfonyl, —S-(2 or 4)-nitrobenzene sulfonyl, —S-m ethanesulfonyl, —S-trifluoromethanesulfonyl, or —S-5(dimethylamino) naphthalene-1-sulfonyl.

In certain embodiments, the kit may further comprise a compound disclosed herein having a hydroxy or thiol and an activating agent such as p-toluenesulfonyl chloride, p-bromobenzenesulfonyl chloride, (2- or 4)-nitrobenzene sulfonyl chloride, methanesulfonyl chloride, trifluoromethanesulfonyl chloride, 5(dimethylamino)naphthalene-1-sulfonyl chloride, dicyclohexylcarbodiimide, bromo-tripyrrolidino-phosphonium hexafluorophosphate, bromotris(dimethylamino) phosphonium hexafluorophosphate, 2-(6-Chloro-1H-benzotriazol yl)-N,N,N′,N′-tetramethylaminium hexafluorophosphate, N-[(5-Chloro-1H-benzotriazol-1-yl)-dimethylamino-morpholino]-uronium hexafluorophosphate N-oxide, tetramethylfluoro formamidinium hexa-fluorophosphate, 1-[1-(cyano-2-ethoxy-2-oxoethylidene-aminooxy)-dimethylamino-morpholino]-uronium hexafluorophosphate, 2-(1-oxy-pyridin-2-yl)-1,1,3,3-tetramethyl-isothiouronium tetrafluoroborate] and a solid support. In certain embodiments, the solid support optionally comprises ¹⁸F salts.

In some embodiments, the solid support is selected from the group of solid phase extraction resins or liquid chromatography resins, e.g., silica (oxide) based or non-silica (metal oxide or polymers) based particles optionally functionalized (e.g., by organosilanization) with alkyl chains for example C₄, C₈, C₁₈, C₃₀ or other functional groups, e.g., polar groups (amide, carbamate, sulfamide, and ureas) embedded within alkyl chains or branched alkyl groups or polymeric packings.

In some embodiments, the solid support is selected from the group consisting of solid phase extraction resins and liquid chromatography resins resulting from the copolymerization of divinylbenzene and/or styrene, or by the copolymerization with vinylpyrrolidone, vinylacetate, (methacryloyloxymethyl)naphtalene, 4,4′-bis(maleimido)diphenylmethane, p,p′-dihydroxydiphenylmethane diglycidylmethacrylic ester, p,p′-dihydroxydiphenylpropane diglycidylmethacrylic ester, 2-hydroxyethylmethacrylate (HEMA), 2,2-dimethylaminoethylmethacrylate (DMAEMA), ethylenedimethacrylate glycidylmethacrylate, N-vinylcarbazole, acrylonitrile, vinylpyridine, N-methyl-N-vinylacetamide, aminostyrene, methylacrylate, ethylacrylate, methylmethacrylate, N-vinylcaprolactam, N-methyl-N-vinylacetamide.

In some embodiments, the solid support comprises or is functionalized with or preconditioned with quaternary ammonium salts, e.g., tetraethylammonium carbonate, tetrabutylammonium carbonate or potassium carbonate cryptands such as 1,4,10-trioxa-7,13-diaza-cyclopentadecane, 4,7,13,16,21,24-hexaoxa-1,10-diazabicyclo[8.8.8]hexacosane, 4,7,13,16,21-pentaoxa-1,10-diazabicyclo[8.8.5]tricosane, 4,7,13,18-tetraoxa-1,10-diazabicyclo[8.5.5] eicosane, 5,6-benzo-4,7,13,16,21,24-hexaoxa-1,10-diazabicyclo[8.8.8] hexacos-5-ene; including crown ethers such as for example 4′-aminobenzo-15-crown-5, 4′-aminobenzo-15-crown-5, 4′-aminobenzo-15-crown-5 hydrochloride, 4′-aminobenzo-18-crown-6, a′-Aminodibenzo crown-6, 2-aminomethyl-15-crown-5, 2-aminomethyl-15-crown-5, 2-aminomethyl-18-crown-6, 4′-amino-5′-nitrobenzo-15-crown-5, 4′-amino-5′-nitrobenzo-15-crown-5, 1-aza-12-crown-4, 1-aza-15-crown-5, 1-aza-15-crown-5, 1-aza-18-crown-6, 1-aza-18-crown-6, benzo-12-crown-4, 5,6-benzo-4,7,13,16,21,24-hexaoxa-1,10-diazabicyclo[8.8.8]hexacos-5-ene, 1-benzyl-1-aza-12-crown-4, bis[(benzo-15-crown-5)-15-ylmethyl]pimelate, 4′-bromobenzo-15-crown-5, 4-tert-butylbenzo-15-crown-5, 4-tert-butylcyclohexano-15-crown-5, 4′-carboxybenzo-15-crown-5, polyethylene glycols (PEG), polyethylene oxides (PEO); the group of calixarenes such as for example 4-tert-butylcalix[4]arene, 4-tert-butylcalix[4]arene, 4-tert-butylcalix[4]arene, 4-tert-butylcalix[5]arene, 4-tert-butylcalix[6]arene, 4-tert-butylcalix[6]arene, 4-tert-butylcalix[6]arene, 4-tert-butylcalix[8]arene, 4-tert-butylcalix[8]arene, 4-tert-butylcalix[4]arene-tetraacetic acid tetraethyl ester, 4-tert-butylcalix[4]arenetetraacetic acid tetraethyl ester, 4-tert-butylcalix[4]arene-tetraacetic acid triethyl ester, calix[4]arene, calix[6]arene, calix[8]arene, 4-(chloromethyl)calix[4]arene, 4-isopropylcalix[4]arene, C-methylcalix[4]resorcinarene, C-methylcalix[4]resorcinarene, meso-octamethylcalix(4)pyrrole, 4-sulfocalix[4]arene, 4-sulfocalix[4]arene sodium salt, C-undecylcalix[4]resorcinarene monohydrate, C-undecylcalix[4]resorcinarene monohydrate, the group of cyclodextrines such as α-cyclodextrin, β-cyclodextrin, γ-cyclodextrin, (2,6-di-O-)ethyl-β-cyclodextrin, 6-O-α-D-glucosyl-β-cyclodextrin, heptakis(6-O-t-butyl dimethylsilyl-2,3-di-O-acetyl)-O-cyclodextrin, heptakis(2,6-di-O-methyl)-O-cyclodextrin, heptakis(2,3,6-tri-O-acetyl)-O-cyclodextrin, heptakis(2,3,6-tri-O-benzoyl)-O-cyclodextrin, hexakis (6-O-tertbutyl-dimethyl silyl)-α-cyclodextrin, hexakis (2,3,6-tri-O-acetyl)-α-cyclodextrin, hexakis (2,3,6-tri-O-methyl)-α-cyclodextrin, (2-hydroxyethyl)-β-cyclodextrin, 6-O-α-maltosyl-β-cyclodextrin hydrate, methyl-β-cyclodextrin, 6-monodeoxy-6-monoamino-β-cyclodextrin, octakis (6-O-t-butyldimethylsilyl)-γ-cyclodextrin, sulfopropyl-O-cyclodextrin, triacetyl-α-cyclodextrin, triacetyl-β-cyclodextrin; and the group of EDTA and derivatives such as for example ethylenediamine-N,N′-diacetic acid, 2-bis(2-aminophenoxy)ethane-N,N,N′,N′-tetraacetic acid, trans-1,2-diaminocyclohexane-N,N,N′,N′-tetraacetic acid monohydrate, trans-1,2-diaminocyclohexane-N,N,N′,N′-tetraacetic acid monohydrate, 1,3-diamino-2-hydroxypropane-N,N,N,N′-tetraacetic acid, 1,2-diaminopropane-N,N,N′,N′-tetraacetic acid, 1,3-diaminopropane-N,N,N′,N′-tetraacetic acid, 1,3-diamino propanol-N,N,N′,N′-tetraacetic acid, diethylenetriamine-pentaacetic acid calcium trisodium salt hydrate, N-(2-hydroxyethyl)ethylenediaminetriacetic acid trisodium salt hydrate, N-(2-hydroxyethyl)ethylenediamine-N,N′,N′-triacetic acid.

### Formulations

In certain embodiments, this disclosure relates to pharmaceutical compositions comprising a compound disclosed herein and a pharmaceutically acceptable excipient. In certain embodiments, the disclosure relates to pharmaceutical compositions comprising a compound disclosed herein and a pharmaceutically acceptable excipient optionally comprising another active agent such as an anticancer agent. In certain embodiments, the disclosure relates to a pharmaceutical composition comprising a compound as described herein including salts and prodrugs thereof and a pharmaceutically acceptable excipient, diluent, or carrier. In certain embodiments, the pharmaceutical composition is in the form of a tablet, pill, capsule, gel, aqueous buffered solution. In certain embodiments, the buffered solution is a citrate buffered solution, isotonic solution, sterile solution, pyrogen free solution, endotoxins and exotoxins free solution, lipopolysaccharide free solution, and/or bacterial free solution.

Pharmaceutical compositions disclosed herein may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid, ascorbic acid and citric acid, as well as other pharmaceutically acceptable acids known per se.

When the compounds of the disclosure contain an acidic group as well as a basic group, the compounds of the disclosure may also form internal salts, and such compounds are within the scope of the disclosure. When a compound contains a hydrogen-donating heteroatom (e.g. NH), salts are contemplated to covers isomers formed by transfer of said hydrogen atom to a basic group or atom within the molecule.

Pharmaceutically acceptable salts of the compounds include the acid addition and base salts thereof. Suitable acid addition salts are formed from acids. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier that releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

Pharmaceutical compositions for use in the present disclosure typically comprise an effective amount of a compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is again made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The compounds can be administered by a variety of routes including the oral, ocular, rectal, transdermal, subcutaneous, intravenous, intratumoral, intramuscular or intranasal routes, depending mainly on the specific preparation used.

### Stereoisomers of 1-Amino-3,4-Difluorocyclopentane-1-Carboxylic Acid (3,4-DFACPC) as PET Imaging Agents

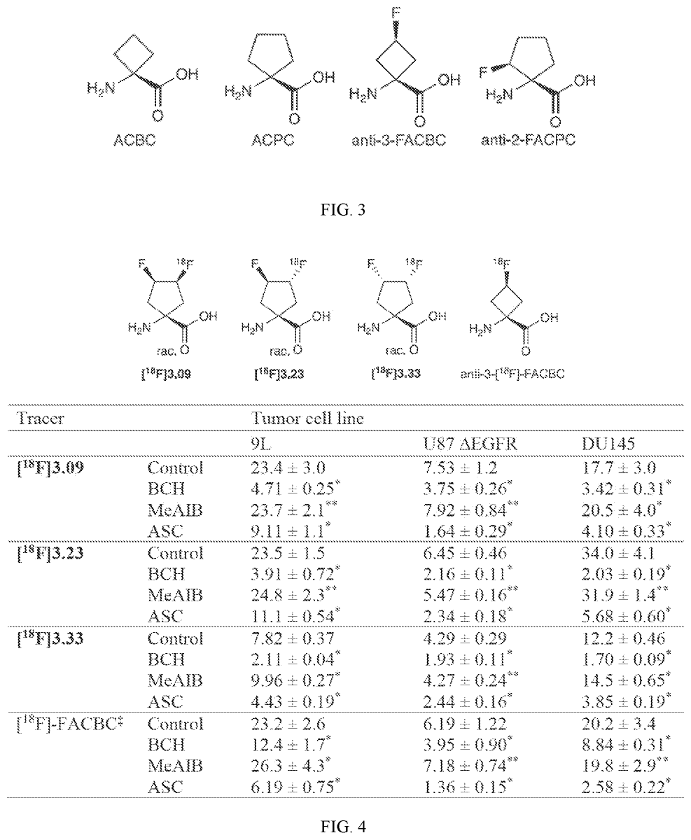

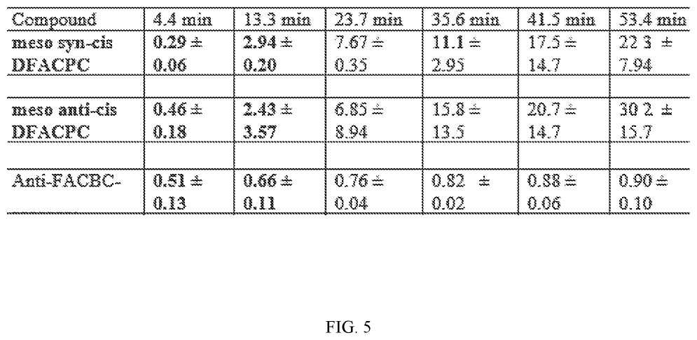

Imaging agent used in studies reported herein are illustrated in FIG. 3. Anti-2-[¹⁸F]fluoro-1-amino-cyclopentane carboxylic acid (anti-2-[¹⁸F]FACPC) and anti-3-[¹⁸F]fluoro-1-amino-cyclobutane carboxylic acid (anti-3-[¹⁸F]FACBC) have been evaluated in a humans with a variety of different types of tumor including breast, lung, prostate, and brain cancer. Relative to [¹¹C]MET-PET, anti-3-[¹⁸F]FACBC-PET shows similar uptake in a variety of tumors, but gives lower background uptake in healthy brain, which may be explained by the fact that it is not metabolized and therefore accumulates in healthy tissues to a lesser extent.

Racemic anti-2-[¹⁸F]fluoro-1-amino-cyclopentane carboxylic acid (anti-2-[¹⁸F]FACPC) is a slightly bulkier analogue of anti-3-[¹⁸F]FACBC. In rats bearing intracranial gliosarcoma, anti-2-[¹⁸F]FACPC and anti-3-[¹⁸F]FACBC showed similar tumor uptake. The relatively lower uptake of anti-2-[¹⁸F]FACPC in healthy brain tissue resulted in T/N ratios of 12:1 which is an approximate two-fold increase over the T/N ratios obtained with anti-3-[¹⁸F]FACBC suggesting that anti-2-[¹⁸F]FACPC may provide a greater degree of differentiation between brain tumors and normal tissue and potentially allowing for more accurate detection and localization of lesions. However, the biodistribution of anti-2-[¹⁸F]FACPC is not ideal for whole body imaging. Rapid accumulation in the bladder is observed and would complicate the detection of tumors near the genitourinary tract, such as primary prostate cancer. Both anti-2-[¹⁸F]FACBC and 2-[¹⁸F]FACPC concentrate in the bladder at early time points in the PET scan, while anti-3-[¹⁸F]FACBC accumulates in the bladder much more slowly and to a lesser extent.

The difluorinated derivative 1-amino-3,4-difluorocyclopentane-1-carboxylic acid (3,4-DFACPC) exists as a pair of achiral cis-difluoro meso diastereomers, in addition to a set of trans difluoro enantiomers. In order to identify compound having a more desirable biodistribution profile, i.e., with less rapid urinary excretion the synthesis of the stereoisomers of [¹⁸F]3,4-DFACPC were prepared and investigated.

### Synthesis of N-benzoyl protected ethyl 1-amino-cyclopent-3-ene-1carboxylate

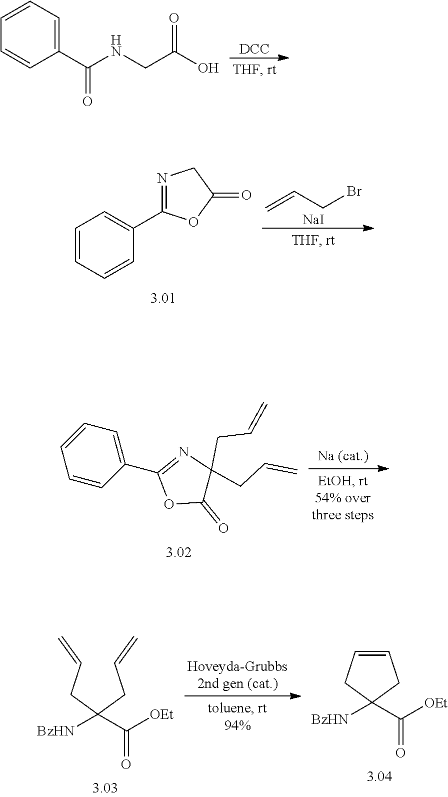

Ethyl 1-amino-cyclopent-3-ene-1-carboxylate could provide access to all four stereoisomers of 3,4-DFACPC, proceeding through a route that makes use of an SN2 displacement of a pseudo halide in the penultimate step. Protecting groups are cleaved under acidic conditions. N-benzoyl protected derivatives were prepared.

Hippuric acid was dehydrated with N,N′-dicyclohexylcarbodiimide to give 3.01, which underwent nucleophilic addition to two equivalents of allyl bromide to give di-allylated intermediate 3.02. Treatment with sodium ethoxide cleaved the oxazolone moiety, resulting in the formation of an N-benzoyl, ethyl ester protected amino acid 3.03 in 54% yield over three steps. The cyclopentene moiety was established with a ring-closing Grubbs metathesis, furnishing 3.04 in 94% yield.

### Synthesis of 3.09 Cold Standard

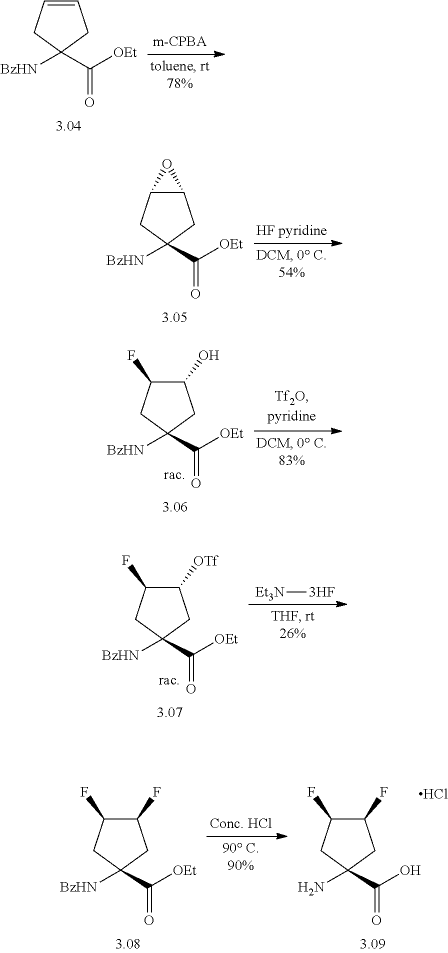

Oxidation (m-CPBA) of 3.04 provided a 9:1 mixture of epoxide diastereomers, favoring the syn-epoxide (3.05). The diastereomers were easily separable by column chromatography and 3.06 was obtained in 78% yield. Fluorination of 3.05 with HF-pyridine resulted in the formation of the desired racemic fluorohydrin 3.06 in 54% yield. Both fluorohydrin enantiomers will be converted to the same C2 symmetric difluoride product, so there is no incentive to pursue an enantioselective fluorination of 3.05. The hydroxyl moiety of 3.06 was converted to triflate 3.07 with triflic anhydride in 83% yield. Triethylamine trihydrofluoride proceeded to give the C2 symmetric difluoride 3.08 in 26% yield. Finally, the benzoyl and ethyl ester protecting groups were removed via acidic hydrolysis at 90° C. with concentrated HCl. When the reaction mixture was allowed to cool to room temperature, anti-cis-3,4-DFACPC (3.09) crystallized spontaneously in 90% yield.

### Conversion of N-Benzoyl Fluorohydrin 3.06 to N-Boc Protected Fluorohydrin 3.12

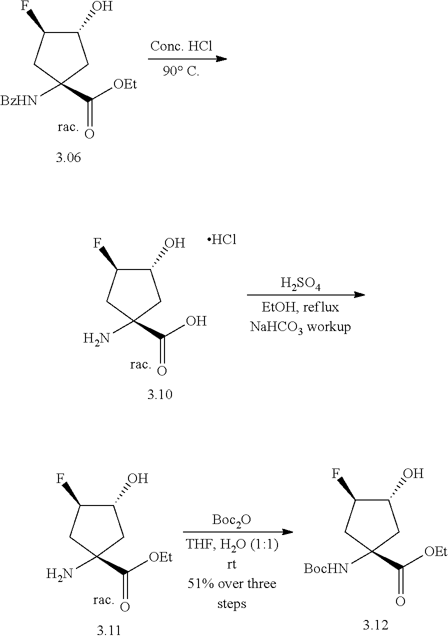

The benzoyl moiety was replaced with a more labile t-butyl carbamate N-protecting group. This manipulation proceeded over three steps, beginning with acidic hydrolysis of the amine and carboxylate protecting groups to give the free amino acid hydrochloride 3.10. The ethyl ester was then reinstalled under Fischer conditions, and the resultant amine 3.11 was treated with di-tert-butyl dicarbonate, furnishing 3.12 in 51% yield over the course of the three-step sequence.

### Synthesis of Syn-Epoxide 3.17 and Anti-Epoxide 3.18

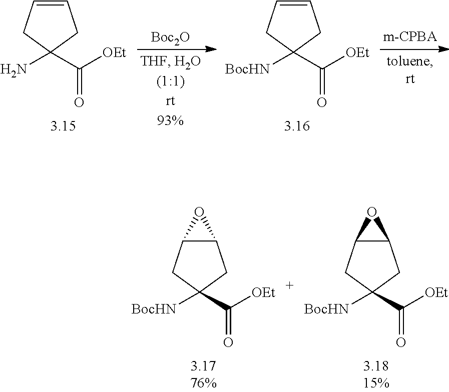 [SMILES: "Boc2O" ; CCOC(=O)C1(N)CC=CC1 ; CCOC(=O)C1(NC(=O)OC(C)(C)C)CC=CC1 ; CCOC(=O)[C@@]1(NC(=O)OC(C)(C)C)C[C@@H]2O[C@@H]2C1 ; CCOC(=O)[C@]1(NC(=O)OC(C)(C)C)C[C@@H]2O[C@@H]2C1]

Compound 3.15 was prepared as reported by Park et al. Diastereoselective Synthesis of Cyclopentanoids with Hydantoin and Isoxazoline Substituents. The Journal of Organic Chemistry 1998, 63, 113-117. N-Boc protection of this compound gave cyclopentene 3.16, which, much like 3.04, underwent oxidation with m-CPBA favoring the formation of the syn-epoxide by a ratio of 5:1. Again, the epoxide diastereomers were easily separable by chromatography and 3.17 was isolated in 76% yield.

Synthesis of Triflate Precursor (3.19) to [¹⁸F]3.09 (Anti-Cis-3,4-[¹⁸F]-DFACPC)

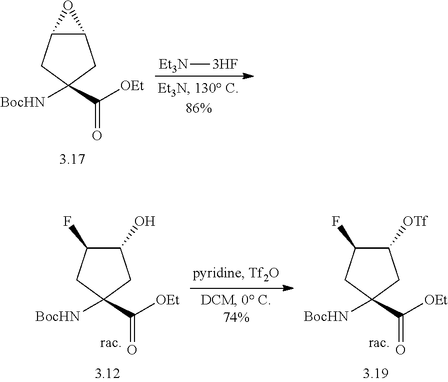

In contrast to benzamide 3.05, which was fairly robust to acidic conditions, the N-Boc bearing compound 3.17 generated complex mixtures on addition of HF-pyridine or triethylamine trihydrofluoride, presumably due to the lability of the Boc group. However, fluorination with triethylamine trihydrofluoride proceeded well with the aid of two equivalents of triethylamine, providing fluorohydrin 3.12 in 86% yield. This route, which proceeded in six steps from glycine ethyl ester hydrochloride and provided 3.12 in 38% yield over six steps, constitutes a substantial improvement over the previous route, which afforded 3.12 in 11% yield over 10 steps.

Compound 3.12 was converted to the triflate, though this was not as straightforward as the analogous conversion of 3.06. On addition of triflic anhydride to 3.06, TLC analysis indicated that reaction had gone to completion within minutes at 0° C., and the Rf of the primary spot as determined by charring with KMnO₄ was consistent with the formation of the triflate 3.19. Because the triflate may be unstable to aqueous conditions, the reaction mixture was pushed directly through a silica plug. The white solid thus obtained gave a proton NMR consistent with loss of the Boc moiety, indicating that if 3.19 was produced, it likely decomposed via an acid mediated process.

The same procedure was attempted with a silica plug that had been pretreated with a 1% triethylamine in DCM, though the same white solid was obtained. The compound was precipitated by diluting the reaction media with hexanes prior to chromatography. On completion of the triflation reaction as determined by TLC, the addition of hexanes caused a white solid to precipitate. After filtration and chromatographic purification of the resultant supernatant, 3.19 was isolated in 74% yield. 3.19 is the direct precursor to ¹⁸F radionuclide incorporation to generate [¹⁸F]3.09 (anti-cis-3,4-[¹⁸F]-DFACPC).

### Synthesis of Triflate Precursor 3.21 and Racemic Trans-3,4-DFACPC (3.23)

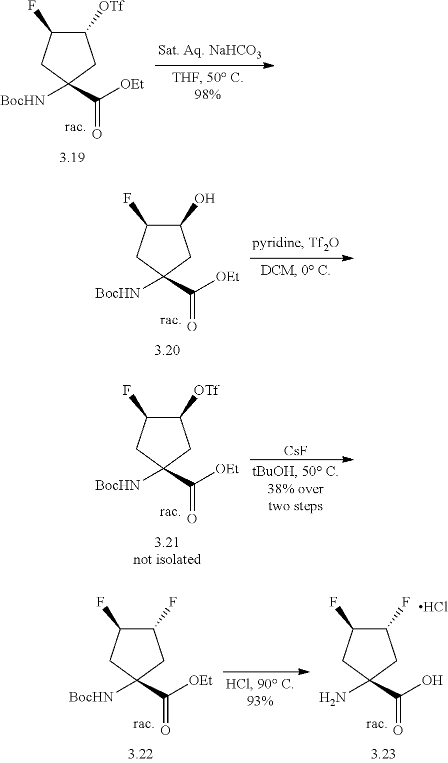

Compound 3.19 was taken up in THF and treated with saturated aqueous sodium bicarbonate, resulting in clean SN2 inversion hydrolysis of the triflate to give racemic fluorohydrin 3.20 in nearly quantitative yield. Compound 3.20 was converted to racemic triflate 3.21 utilizing the same procedure described for 3.19, though unlike 3.19, 3.21 is highly unstable and was not purified by chromatography but used directly in subsequent fluorination reactions. Our attempts to fluorinate 3.21 were unsuccessful under a variety of conditions, owing to the formation of olefinic byproducts via elimination of the triflate. Ultimately, a particularly mild method which involves the use of cesium fluoride in tertiary alcohol solvents, proved to be an efficient method for the introduction of fluoride and racemic trans-difluoride 3.22 was generated in 38% yield over two steps from 3.20.

Compound 3.22 was then treated with concentrated HCl at 90° C. to remove the Boc and ethyl ester protecting groups, giving racemic difluoride 3.23 (trans-3,4-DFACPC) in 93% yield. As was the case with 3.09, crystals of 3.23 grew spontaneously from the reaction mixture when it was cooled from 90° C. to room temperature, and an X-ray crystal structure was obtained.

Attempted Synthesis of N-Boc Protected Syn-Cis-3,4,-DFACPC from Triflate Precursor 3.25

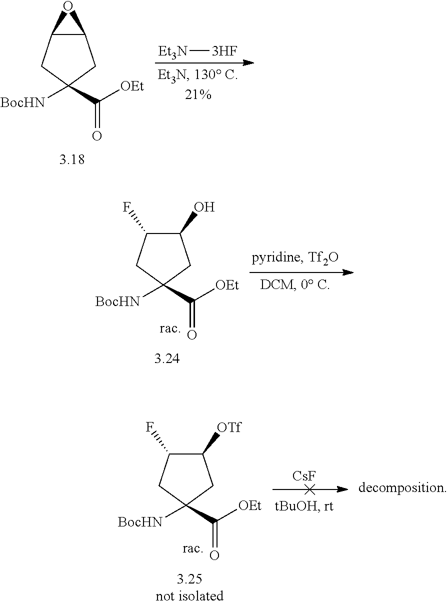

Compound 3.18 was treated with triethylamine trihydrofluoride to afford 3.24 in 21% yield. Compound 3.24 was treated with triflic anhydride to furnish the highly unstable triflate 3.25, which could not be isolated. Attempts to displace the triflate moiety of 3.25 with fluoride met with failure. Crude NMR and LCMS analysis of these failed reactions was consistent with the formation of a byproduct in which the Boc and triflate moieties had been lost.

### Synthesis of Phthalimide Protected Syn-Cis-3,4-DFACPC (3.31)

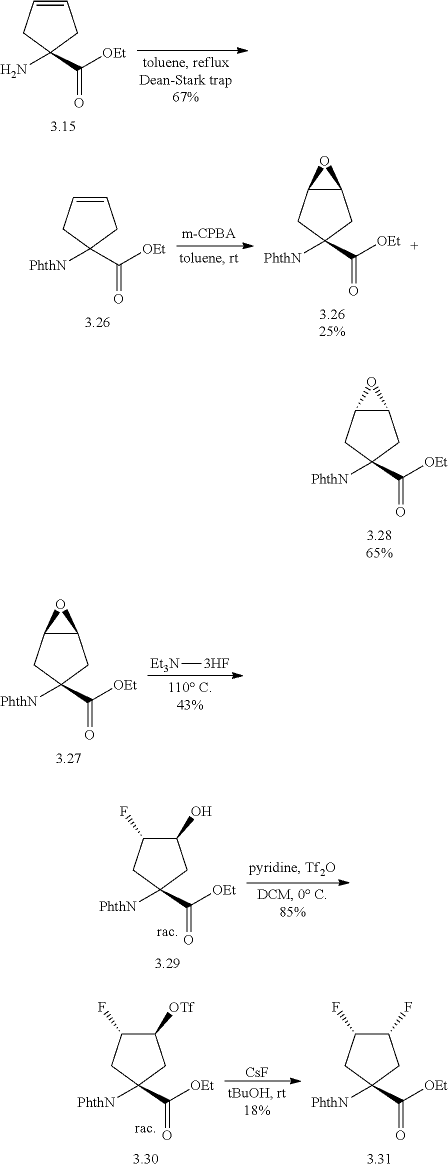

Compound 3.15 and phthalic anhydride were combined in a Dean-Stark apparatus with refluxing toluene resulting in the formation of phthalimide 3.26 in 67% yield. Epoxidation of 3.26 with m-CPBA was slightly more selective for the anti-epoxide than it was in the case of 3.04 and 3.16, resulting in a 2.6:1 mixture of syn and anti-epoxide diastereomers, respectively. However, in contrast to the epoxide diastereomers described earlier, 3.27 and 3.28 were quite challenging to separate by chromatography, though they could be obtained in pure form by crystallization. Compound 3.27 thus obtained was treated with triethylamine trihydrofluoride to afford racemic fluorohydrin 3.29 in 43% yield. 3.29 was converted to racemic triflate 3.30, which was stable at room temperature and isolated in 85% yield following chromatographic purification. Fluorination of 3.30 proved challenging as elimination byproducts formed preferentially over the desired difluoride. Difluoride 3.31 was obtained in 18% yield utilizing cesium fluoride in tert-butanol.

Two Step Hydrazine, NBS Deprotection Procedure for Generating Syn-Cis-3,4-DFACPC (3.33) from Phthalimide 3.31.

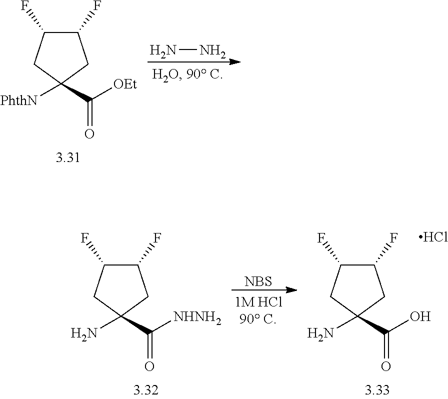

Aqueous hydrazine was used with 3.31 in hand to remove the phthaloyl and ester protecting groups. LCMS analysis of this reaction mixture indicated the formation of one product with a mass that was consistent with the loss of the phthaloyl and ethyl groups, as well as incorporation of hydrazine, suggesting the formation of an amino hydrazide. Hydrazides are reticent to undergo hydrolysis under acidic or basic conditions, though they are labile to oxidation and can be displaced in aqueous media to reveal the parent carboxylic acid. Thus, one pot deprotection of 3.31 was attempted by first treating the compound with aqueous hydrazine to generate the hydrazide, then evacuating the reaction vessel at 100° C. under a flow of inert gas, before finally adding 1M HCl (to protonate the free amine) and N-bromosuccinimide oxidant. The syn-cis-3,4-DFACPC cold standard was generated with this procedure.

### Synthesis of Syn-Cis-3,4-DFACPC (3.33) by Aqueous Hydrolysis of 3.31

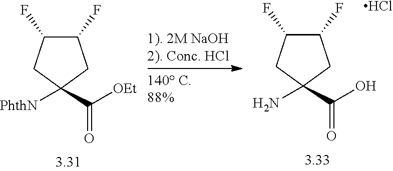

Treating 3.31 with 2M aqueous sodium hydroxide at 140° C. for five minutes, followed by addition of concentrated HCl with heating at 140° C. for another 15 minutes resulted in the clean formation of amino acid 3.33 in 88% yield. The manipulations associated with this protocol proved straightforward enough to be translated to the radiochemistry lab, allowing for the synthesis of syn-cis-3,4-[¹⁸F]-DFACPC.

Synthesis of [¹⁸F]3.09 (Anti-Cis-3,4-[¹⁸F]-DFACPC) from Triflate 3.19.

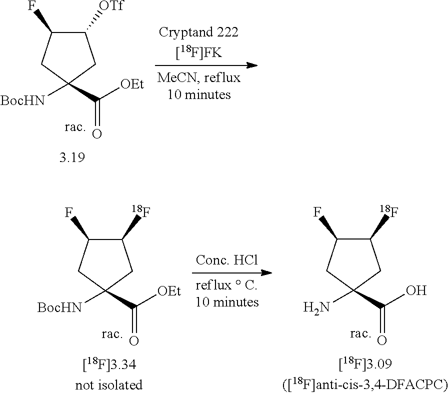 [SMILES: CCOC(=O)[C@@]1(NC(=O)OC(C)(C)C)C[C@@H](F)[C@H](OS(=O)(=O)C(F)(F)F)C1 ; CCOC(=O)[C@@]1(NC(=O)OC(C)(C)C)C[C@@H](F)[C@@H]([18F])C1 ; N[C@]1(C(=O)O)C[C@@H](F)[C@@H]([18F])C1]

Radiosynthesis described herein was carried out in two types of fume hoods for preclinical radiosynthetic use. One hood contains a computer process control unit (CPCU): a programmable system that carries out various steps in the radiolabeling process with 5 vials. The other hood, referred to as a “hot-cell”, is fitted with a pair of mechanical arms that allow laboratory personnel to manually perform synthetic manipulations inside the hood while standing behind a closed leaded door. The hot-cell also contains a dose calibrator (gamma counter) that is used to determine the quantity of radioactivity within a particular vessel. In practice, the CPCU is generally used for the introduction of ¹⁸F−, simple deprotection reactions, and filtration of the labeled compound through various adsorbents, while more complicated synthetic procedures, purification steps involving fraction collection, and preparation of doses are handled in the hot-cell.

Prior to the start of the synthesis, each vial in the CPCU was charged with the necessary mixture of solutions and reagents and equipped with a silicone rubber septum secured with an aluminum crimp top, an inlet line for inert nitrogen gas to pressurize the vial, and Teflon outlet lines for transfer of the contents of the vials to the reaction vessel or to the ion exchange “trap and release” cartridge. The reaction vessel was equipped similarly to allow for transfer of its contents to the ion retard resin, alumina, and Oasis HLB cartridge chain used for purification. Approximately 790 mCi of ¹⁸F as [¹⁸F]HF was transferred from the cyclotron to the trap and release cartridge, and an aqueous solution of potassium carbonate from vial 5 was flushed through the cartridge to generate an aqueous solution of [¹⁸F]KF, which eluted into the reaction vessel. A solution of Cryptand 222 in acetonitrile from vial 1 was then transferred to the reaction vessel, and the vessel was heated with an oil bath under a flow of inert gas to evaporate the acetonitrile with azeotropic removal of water. A second aliquot of acetonitrile (from vial 2) was added and evaporated to ensure that the contents of vessel 1 were free of residual water. A solution containing 9 mg (0.021 mmol) of triflate precursor 3.19 in acetonitrile was then transferred to the reaction vessel from vial 3, and the reaction was heated to reflux for 10 minutes. The reaction was terminated after 10 minutes by the evaporation of acetonitrile under inert gas flow. A 6M solution of HCl was transferred to vessel 1 from vial 4 and heated for 10 minutes to cleave the N-Boc and ethyl ester protecting groups, giving crude [¹⁸F]3.09. The contents of vessel 1 were then pushed through ion retard resin, alumina, and Oasis HLB (reverse phase) cartridges, and vessel 1 was rinsed with an aliquot of saline that was also passed through the chain of adsorbent containing cartridges to ensure that all of the [¹⁸F]3.09 was collected and eluted. The line carrying the eluent terminated inside the hot-cell where fractions were collected manually, and the most concentrated fractions were used as doses for in vivo and in vitro use.

The identity of the radioactive species in the dose vials was assayed by comparison of the Rf (on silica) of the ¹⁸F labeled material to the authentic cold racemic 3.09. Because the concentration of ¹⁸F is low (1 Ci of ¹⁸F is approximately 80 nanomoles), the direct detection of [¹⁸F]3.09 by UV or staining of the TLC plate was not feasible. Instead, a radiometric TLC scanner was used to determine the Rf of the radioactive compounds on the silica TLC plate. The radiometric TLC chromatogram showed a small peak at the baseline consistent with residual ¹⁸F, and a much larger peak with a Rf value consistent with that of the authentic 3.09 standard, indicating that [¹⁸F]3.09 was obtained in >99% radiochemical purity.

The compound [¹⁸F]3.09 (50 mCi) was obtained in 12 mL of saline solution affording a decay corrected yield of 10%. The specific activity, a measure of the quantity of radioactivity arising from a particular compound in a sample of a given mass, was estimated to be no less than 2.4 Ci/mmol based on the assumption that all of the starting precursor (which was used in approximately 300 fold excess relative to [¹⁸F]CsF) that was not converted to [¹⁸F]3.09 remained in the dose as a non-radioactive amino acid byproduct. The concentration of the dose is significant because the sample volume that can be administered in both in vitro and in vivo assays is finite, and a dose that is not sufficiently concentrated will prevent the study from proceeding with the intended quantity of radioactive compound.

Synthesis of racemic [¹⁸F]3.23 (Trans-3,4-[¹⁸F]-DFACPC)

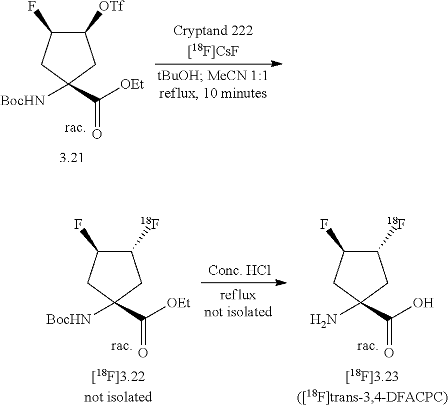 [SMILES: CCOC(=O)[C@@]1(NC(=O)OC(C)(C)C)C[C@@H](F)[C@@H](OS(=O)(=O)C(F)(F)F)C1 ; CCOC(=O)[C@@]1(NC(=O)OC(C)(C)C)C[C@@H](F)[C@H]([18F])C1 ; N[C@]1(C(=O)O)C[C@@H](F)[C@H]([18F])C1]

The synthesis of racemic [¹⁸F]3.23 (trans-3,4-[¹⁸F]-DFACPC) proceeded similarly to [¹⁸F]3.09, with a few changes to the reagents employed in the CPCU. Initially, [¹⁸F]KF was used as the nucleophilic fluoride source, though these reactions were unsuccessful, likely owing to competitive decomposition of the triflate under heating. Triflate 3.21 decomposed much more rapidly than 3.19.

[¹⁸F]CsF in 1:1 tert-butanol/acetonitrile solvent proved to be an effective system for labeling triflate 3.21 (20 mg, 0.047 mmol) to give racemic [¹⁸F]3.23. The CPCU automated synthesis of racemic [¹⁸F]3.23 was comparable to the protocol used to prepare [¹⁸F]3.09; the only notable changes in protocol include the use of cesium carbonate in vial 5, resulting in the formation of [¹⁸F]CsF rather than [¹⁸F]KF, the use of a mixture of 1:1 tert-butanol/acetonitrile solvent in vial 3 rather than pure acetonitrile. After the reaction was carried out and purified as described above, the contents of the dose were compared with authentic 3.23 using chiral analytical HPLC with an inline UV detector and radiation counter. HPLC analysis indicated that [¹⁸F]3.23 was obtained in >99% radiochemical purity. 12 mCi of racemic [¹⁸F]3.23 were obtained in 7 mL of saline solution, affording a decay corrected yield of 1.3%, and the specific activity was estimated to be no less than 0.26 Ci/mmol based.

Synthesis of [¹⁸F]3.33 (Syn-Cis-3,4-[¹⁸F]-DFACPC) from Triflate 3.30

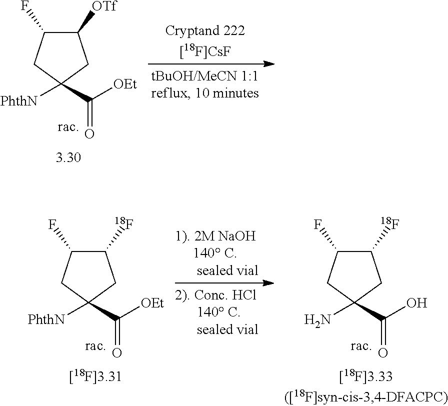

The compound [¹⁸F]3.33 (syn-cis-3,4-[¹⁸F]-DFACPC), proved to be more difficult to prepare and required significant departures from the protocols that were used for the other 3,4-DFACPC stereoisomers owing to the challenges associated with deprotection of the phthalimide moiety. As was the case with triflate 3.21, triflate 3.30 failed to produce the desired labeled amino acid under the typical conditions ([¹⁸F]KF, Cryptand 222, acetonitrile solvent).

Cesium fluoride enabled the fluorination of triflate 3.21, also proved to be effective for the conversion of triflate 3.30 to difluoride 3.31. The CPCU was equipped as follows: an aqueous solution of cesium carbonate in vial 5, Cryptand 222 in acetonitrile in vial 1, acetonitrile in vial 2, triflate 3.30 in a 1:1 mixture of tert-butanol/acetonitrile in vial 3, and another volume of acetonitrile in vial 4. 1460 mCi of [¹⁸F]HF was transferred from the cyclotron onto the trap and release cartridge and washed with the contents of vial 5, generating an aqueous solution of [¹⁸F]CsF in reaction vessel 1. The contents of vial 1 were added to the reaction vessel and the solvent was removed via heating under a flow of inert gas. This drying procedure was repeated after a second aliquot of acetonitrile from vial 2 was added to vessel 1. The tert-butanol/acetonitrile mixture containing triflate 3.30 (20 mg, 0.044 mmol) in vial 3 was then added to vessel 1, and the mixture was heated to reflux for 10 minutes. In contrast to the previous two procedures, the contents of vessel 1 were not concentrated and treated with aqueous HCl at this stage. Instead, the crude mixture containing difluoride [¹⁸F]3.31 was loaded onto a chain of two alumina cartridges. The reaction vessel was rinsed with acetonitrile from vial 4, and this solution was used to elute the remaining material from the alumina cartridges into the hot-cell where fractions were collected manually. The most concentrated fractions were combined in a single v-vial and concentrated at reflux under a flow of inert gas. At this stage, the contents of the reaction mixture were examined by radiometric TLC on silica. Two peaks were observed in the radiometric TLC chromatogram; a small peak at the baseline corresponding to residual unreacted [¹⁸F]CsF, and a second, much larger peak with an Rf consistent with that of the authentic difluoride 3.31 in the same solvent system, verifying that difluoride [¹⁸F]3.31 was prepared.

To deprotect this compound it was reacted with a 1:1 solution of hydrazine and water, assuming that these conditions would generate amino hydrazide [¹⁸F]3.32. After 10 minutes at 90° C., the reaction was concentrated at reflux under inert gas flow and the mixture was treated with 1M HCl to protonate the amine of [¹⁸F]3.32. Excess N-bromo succinimide was then added to the crude [¹⁸F]3.32, and the mixture was heated to reflux for another 10 minutes at 90° C., then cooled to room temperature. Despite the fact that this two-step procedure was able to reliably produce the cold standard 3.33 in pure form, radiometric TLC analysis of the crude mixture showed that multiple [¹⁸F] containing compounds were obtained. Given the time constraints and other difficulties associated with handling [¹⁸F] labeled molecules, purification of the mixture was considered to be infeasible.

Another two-step protocol was tested for the deprotection of [¹⁸F]3.31 involving basic hydrolysis with 2M NaOH followed by acidic hydrolysis with conc. HCl. The hydrolysis reactions required heating at 140° C. in order to proceed on the necessary time scale. Typically, heating solutions beyond their boiling point can be achieved with a sealed tube or other suitable apparatus fitted with an appropriate screw cap that is capable of withstanding high pressures. However, this is not practical in the hot-cell. Screw capped v-vials with thick Teflon coated septa are used prior to the transfer of radioactive material from the CPCU. Solutions of [¹⁸F]3.31 could be syringed into a sealed v-vial, then briefly heated to 140° C. to allow the reaction to occur while the septa remained intact. Thus, once [¹⁸F]3.31 was transferred from the CPCU into the hot-cell as a solution in acetonitrile, it was syringed into a screw capped v-vial fitted with a Teflon septum. The vial was placed in a pie plate maintained at 140° C., fitted with an outlet needle and a second needle connected to an inert gas line, and the acetonitrile solvent was removed under inert gas flow. After evaporation, both needles were removed, 2M NaOH was syringed into the reaction, and the mixture was allowed to stand for 5 minutes at 140° C. After cooling briefly, concentrated HCl was syringed into the reaction vial, and the mixture was heated for 15 minutes at 140° C. then evaporated under inert gas flow (the outlet needle was fitted with a line that was submerged in saturated aqueous potassium carbonate to neutralize the HCl fumes and prevent etching of the leaded glass doors). However, the integrity of Teflon septum was compromised, and it no longer acted as an effective seal, which rendered pressure induced cannulation from the reaction vessel in the following purification steps inefficient and negatively impacted the radiochemical yield. Nonetheless, water was syringed through the septum into the reaction vial to dissolve the crude [¹⁸F]3.33, and the mixture was cannulated under pressure into a chain of Oasis HLB (reverse phase) and alumina cartridges. The eluent was collected in fractions, and the fractions with the greatest concentration of radioactivity were used for in vitro and in vivo studies. The contents of the dose solution were assayed by analytical HPLC, utilizing [¹⁸F]3.23 as a reference standard. HPLC analysis indicated that [¹⁸F]3.33 was obtained in >99% radiochemical purity. 8.4 mCi of [¹⁸F]3.33 were obtained in 5 mL of water, affording a decay corrected yield of 1.7%, and the specific activity was estimated to be no less than 0.19 Ci/mmol.

### Cell Uptake Studies

Amino acids enter cells, both healthy and neoplastic, via functionally and biochemically distinct amino acid transporters (AATs) that are categorized based on their selectivity for particular amino acids as well as their physico-chemical properties. AATs from system L, ASC, and A are the most abundant AATs in the majority of mammalian cells and are overexpressed in a variety of cancers. For example, systems L and ASC are most highly expressed in brain, breast, ovary, lung, liver, pancreas, and prostate cancers while system A is overexpressed in prostate, glioma, hepatocellular carcinoma, hilar cholangiocarcinoma, and breast cancer. Amino acids containing a positron emitting element that have a high affinity for an overexpressed AAT can be used to identify tumors via PET, since the amino acid will tend to concentrate in the tumor to a greater extent than in healthy cells.

Consistent with this reasoning, having knowledge of which systems take part in transporting a particular amino acid is of value, since this information can be used to determine which tumor types the amino acid may be useful for imaging. To establish which systems are responsible for transporting a particular AA, cell uptake inhibition studies are performed. In these studies, a known number of cells of a certain line are suspended in a media containing a known quantity of an amino acid radiotracer and incubated for a set period to allow for the amino acid to be transported into the cells. The cells are then collected, centrifuged, and rinsed to separate them from the remaining tracer that did not undergo cellular transport. The radioactivity present within the cells after rinsing is measured and expressed as a normalized percent uptake of the radioactive amino acid dose that the cells were initially exposed to. This experiment establishes the degree to which a given amino acid is taken up by a group of cells in the absence of any perturbing conditions and serves as the control.

With this information in hand, the cell uptake experiment is performed again, but in the presence of an excess of a substrate known to have a high affinity for a particular transport system. The excess substrate floods the targeted system, competitively inhibiting the transport of other amino acids. Consequently, any reduction in cellular uptake of radioactivity relative to the control experiment is assumed to arise from loss of amino acid transport by the inhibited system. It follows that if inhibition of a transport system results in reduced uptake of a given amino acid, then that system contributes to cellular transport of the amino acid. Furthermore, the degree to which the system participates in transport can be roughly evaluated by the percent loss (inhibition) of amino acid uptake relative to the control. In addition to delineating transport mechanisms, cell uptake studies provide insight into the avidity of a particular cell line for an amino acid.

The potential of [¹⁸F]3.09, [¹⁸F]3.23, and [¹⁸F]3.33 as PET radiotracers were evaluated for imaging brain and prostate cancer, cell uptake studies with rat 9 L gliosarcoma, human U87 ΔEGFR glioblastoma, and human DU145 androgen-independent prostate carcinoma. Uptake data for anti-3-[¹⁸F]-FACBC were also collected under the same conditions for comparison. Alpha-(methylamino)isobutyric acid (MeAIB) was used as a competitive inhibitor for system A transport, 2-amino-bicyclo[2.2.1]heptane2-carboxylic acid (BCH) was used to inhibit system L, and the combination of alanine, serine, and cysteine was used for inhibition of system ASC. See Table in FIG. 4.

The uptake levels of [¹⁸F]3.09, [¹⁸F]3.23, and [¹⁸F]3.33 were relatively high, ranging between approximately 4-34% of the initial dose per 0.5 million cells (% ID/5×10⁵ cells) across all cell lines tested, compared to 6-20% ID/5×10⁵ cells with anti-3-[¹⁸F]-FACBC. [¹⁸F]3.09 and [¹⁸F]3.23 showed greater uptake than [¹⁸F]3.33 in all cell lines, particularly in rat 9 L gliosarcoma and human DU145 androgen-independent prostate carcinoma cells. The uptake of [¹⁸F]3.09 and [¹⁸F]3.23 were similar in rat 9 L gliosarcoma and U87 ΔEGFR glioblastoma cells, and these uptake data were comparable to those obtained for anti-3-[¹⁸F]-FACBC. In DU145 androgen-independent prostate carcinoma cells, the uptake of [¹⁸F]3.23 (34% ID/5×10⁵ cells) was nearly double that of [¹⁸F]3.09 (18% ID/5×10⁵ cells) and was also substantially higher than the uptake of anti-3-[¹⁸F]-FACBC (20% ID/5×10⁵ cells). With regard to transport mechanism, data from each of the three cell lines used in this study demonstrate that [¹⁸F]3.09, [¹⁸F]3.23, and [¹⁸F]3.33 undergo transport predominantly by system L (50-94% inhibition by BCH) with some transport occurring through system ASC (43-83% inhibition by alanine, serine, and cysteine). The lone exception is [¹⁸F]3.09 in U87 ΔEGFR glioblastoma cells, as 78% of uptake was inhibited by alanine, serine, and cysteine in this cell line, compared to 50% inhibition with BCH. MeAIB did not result in significant uptake inhibition for any of the stereoisomers of 3,4-[¹⁸F]-DFACPC. These results are similar to those obtained in 9 L gliosarcoma cells for anti-2-[¹⁸F]-FACPC, which is transported by system L (71% inhibition by BCH) and ASC (65% inhibition by alanine, serine, and cysteine), though it is also transported to a lesser degree by system A (38% inhibition by MeAIB). Anti-3-[¹⁸F]-FACBC undergoes some transport by system L (36-56% inhibition by BCH) in all cell lines, though it is primarily a substrate for system ASC (73-87% inhibition by alanine, serine, and cysteine).

Biodistribution Studies in Normal Fischer Rats and Fischer Rats with Intracranial 9 L Gliosarcoma Tumors

Low levels of radiotracer uptake in normal tissues with comparatively high tumor uptake is desirable, since contrast in uptake between tumors and adjacent normal tissues is the basis for tumor visualization via PET. Therefore, high uptake in normal tissues is problematic. For example, despite being a useful radiotracer for the localization of a wide variety of tumors, [¹⁸F]FDG is not an effective imaging agent for brain and prostate tumors owing to its high uptake in normal brain tissue and in the bladder, which is proximal to the prostate. For tracers intended to image brain tissue, an additional consideration is the permeability of the blood-brain barrier (BBB). Charged small molecules such as amino acids are not able to freely diffuse through the BBB but can be brought into the brain by facilitated transport. Of the ubiquitous AAT systems, only system L is present in the lumen of the brain, thus only radiotracers that undergo system L transport hold promise for imaging intracranial tumors.

To gain insight into the biodistribution profiles of [¹⁸F]3.09, [¹⁸F]3.23, and [¹⁸F]3.33 each was administered to Fischer rats bearing intracranial 9 L gliosarcoma tumors, which have been used extensively as models for human glioma. With each tracer, uptake in 9 L tumors was higher than in normal brain tissue at all time points, peaking near the 12.5-minute time point. Tumor uptake was highest with [¹⁸F]3.09 and stayed fairly constant at 2.0-2.2% injected dose per gram of tissue (% ID/g) throughout the course of the study. Lower uptake was observed with [¹⁸F]3.23 and [¹⁸F]3.33, both of which peaked at 1.2% ID/g, though the concentration of radioactivity in the 9 L tumors fell more rapidly with the latter compound, dropping to 0.8% ID/g by the final time point while uptake of [¹⁸F]3.23 remained nearly constant (1.1% ID/g during the final scan). While absolute uptake in tumors was highest with [¹⁸F]3.09, background uptake in healthy brain was also highest with this stereoisomer, ranging from 0.59-0.96% % ID/g, compared to uptake values of 0.39-0.46 and 0.28-0.36% ID/g obtained with [¹⁸F]3.23 and [¹⁸F]3.33, respectively.

Despite the lower absolute tumor uptake observed with [¹⁸F]3.33, this stereoisomer gave the highest L/N ratio of 4.3 at the 4.5-minute time point, though this value fell in each following time point owing to the progressive loss of activity in the 9 L tumor over the course of the study. The highest L/N ratio obtained with [¹⁸F]3.09 also occurred at the 4.5-minute time point (L/N of 3.5) and similarly decreased over time, though this resulted from accumulation of radioactivity in normal brain rather than loss of activity in the tumor. [¹⁸F]3.23 reached a maximum at the 12.5-minute mark (L/N of 2.8), and its concentration in the 9 L tumor and normal brain remained steady over the course of the study (L/N of 2.4 at 52.5 minutes). These uptake profiles are consistent with system L and ASC mediated transport. Because these systems transport via exchange rather than unidirectional flow of amino acid substrates into the cell, it is often the case that system L and ASC substrates will reach their peak concentration in tissues at early time points and remain relatively steady, or slowly decrease with time. Nonetheless, the relatively high absolute uptake of [¹⁸F]3.09, [¹⁸F]3.23, and [¹⁸F]3.33 in 9 L tumors coupled with low background uptake in normal brain suggests that these compounds may have promise for imaging glioma.

Cell uptake levels were relatively high for each stereoisomer in each of the cell lines tested, which included 9 L gliosarcoma, human U87 ΔFGFR glioblastoma, and human DU145 androgen-independent prostate carcinoma, though [¹⁸F]3.09 and [¹⁸F]3.23 were taken up to a greater extent than [¹⁸F]3.33 in all cell lines. DU145 cells displayed a substantially greater avidity for [¹⁸F]3.23 than for the other compounds tested. These data suggest that [¹⁸F]3.23 is promising for imaging prostate cancer.

### Racemic ethyl-1-benzamido-3-fluoro-4-(((trifluoromethyl)sulfonyl)oxy)cyclopentane carboxylate (3.07)

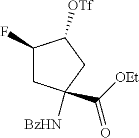

A scintillation vial under N₂ containing a stir bar and racemic ethyl-1-benzamido-3-fluoro-4-hydroxycyclopentane-1-carboxylate (3.06) (400 mg, 1.35 mmol, 1 equiv) and pyridine (240 μL, 2.97 mmol, 2.2 equiv) in DCM (5 mL) was cooled to 0° C. A separate vial containing trifluoromethanesulfonic anhydride (385 μL, 2.70 mmol, 2.0 equiv) in DCM (1.5 mL) was cooled to 0° C., and this mixture was added dropwise to the fluorohydrin solution with vigorous stirring. The mixture was stirred at 0° C. for 15 minutes, then diluted with hexanes (10 mL). A white powder precipitated and was filtered away, and the supernatant was concentrated at 0° C. The crude residue was purified with a silica plug, eluting the desired compound with a 30/70 EtOAc/hexanes gradient (R_{f}=0.3). Colorless oil, 481 mg, 1.13 mmol, 83% yield. Note, these compounds are thermally unstable. They should be isolated from the reaction mixture as quickly as possible and used immediately, or stored as a solution in benzene at <0° C. ¹HNMR (400 MHz, chloroform-d) δ 7.78-7.74 (m, 2H), 7.57-7.51 (m, 1H), 7.48-7.43 (m, 2H), 5.62 (ddd, J=8.4, 6.3, 5.2 Hz, 1H), 5.50 (dtd, J=15.7, 7.8, 5.3 Hz, 1H), 4.34 (qd, J=7.1, 4.7 Hz, 2H), 2.90-2.78 (m, 3H), 2.51 (ddd, J=23.3, 15.0, 6.5 Hz, 1H), 1.34 (t, J=7.1 Hz, 3H). ¹³C NMR (100 MHz, chloroform-d) δ 172.8, 166.9, 133.5, 132.4, 129.0, 118.6 (q, J=320 Hz), 94.1 (d, J=188 Hz), 89.8 (d, J=25 Hz), 63.3, 61.9 (d, J=5.0 Hz), 39.5 (d, J=22 Hz), 38.2 (d, J=3.4 Hz), 14.1. ¹⁹F NMR (376 MHz, chloroform-d, fluorobenzene reference standard) δ −74.5 (s, 3F), −184.7 (ddd, J=53.6, 23.7, 12.4 Hz, 1F). IR (neat, cm⁻¹): 3301, 1737, 1636. HRMS (ESI) Calcd. for C₁₆H₁₈O₆NF₄S (M+H)⁺: 428.07855. Found: 428.07894.

### Racemic ethyl-1-((tert-butoxycarbonyl)amino)-3-fluoro-4 (((trifluoromethyl)sulfonyl)oxy) cyclopentane-1-carboxylate (3.19)

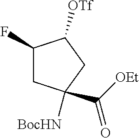 [SMILES: CCOC(=O)[C@@]1(NC(=O)OC(C)(C)C)C[C@@H](F)[C@H](OS(=O)(=O)C(F)(F)F)C1]

A scintillation vial under N₂ containing a stir bar and a mixture of racemic 1-((tert-butoxycarbonyl)amino)-3-fluoro-4-hydroxycyclopentane-1-carboxylate (3.12) (70 mg, 0.24 mmol, 1 equiv) and pyridine (40 μL, 0.53 mmol, 2.2 equiv) in DCM (1.5 mL) was cooled to 0° C. A separate vial containing trifluoromethanesulfonic anhydride (70 μL, 0.48 mmol, 2.0 equiv) in DCM (1.5 mL) was cooled to 0° C., and this mixture was added dropwise to the fluorohydrin solution with vigorous stirring. The mixture was stirred at 0° C. for 15 minutes, then diluted with hexanes (3 mL). A white powder precipitated and was filtered away, and the supernatant was concentrated at 0° C. The crude residue was purified with a silica plug, eluting the desired compound with a 20/80 EtOAc/hexanes gradient (R_{f}=0.3). Colorless oil, 75 mg, 0.177 mmol, 74% yield. Note, these compounds are thermally unstable, decomposing over the course of one evening at room temperature. Care should be taken to purify these compounds as quickly as possible, at which point they should be used immediately, or taken up in benzene and stored at <0° C. ¹H NMR (600 MHz, chloroform-d) δ 5.54-5.25 (m, 3H), 4.27 (q, J=7.1 Hz, 2H), 2.95-2.35 (m, 4H), 1.44 (d, 9H), 1.31 (t, J=7.1 Hz, 3H). ¹³C NMR (100 MHz, chloroform-d) δ 172.4, 154.6, 118.6 (q, J=319 Hz), 94.6 (d, J=188 Hz), 92.2, 89.9 (d, J=26 Hz), 62.8, 58.1, 40.2 (d, J=22 Hz), 39.2, 28.4, 14.1. ¹⁹F NMR (376 MHz, chloroform-d, fluorobenzene reference standard) δ −74.6 (s, 3F), −184.6 (m, 1F). IR (CDCl₃, cm⁻¹): 3353, 1716. HRMS (ESI) Calcd. for C₁₄H₂₂O₇NF₄S (M+H)⁺: 424.10476. Found: 424.10539.

### Racemic ethyl-1-((tert-butoxycarbonyl)amino)-3-fluoro-4-(((trifluoromethyl)sulfonyl)oxy) cyclopentane-1-carboxylate (3.21)

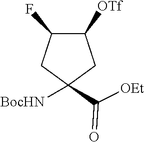 [SMILES: CCOC(=O)[C@@]1(NC(=O)OC(C)(C)C)C[C@@H](F)[C@@H](OS(=O)(=O)C(F)(F)F)C1]

A scintillation vial under N₂ containing a stir bar and racemic ethyl 1-((tert-butoxycarbonyl)amino)-3-fluoro-4-hydroxycyclopentane-1-carboxylate (3.20) (50 mg, 0.17 mmol, 1 equiv) and pyridine (30 μL, 0.38 mmol, 2.2 equiv) in DCM (0.5 mL) was cooled to 0° C. A separate vial containing trifluoromethanesulfonic anhydride (50 μL, 0.34 mmol, 2.0 equiv) in DCM (0.5 mL) was cooled to 0° C., and this mixture was added dropwise to the fluorohydrin solution with vigorous stirring. The mixture was stirred at 0° C. for 15 minutes, then diluted with hexanes (1 mL). A white powder precipitated and was filtered away, and the supernatant was concentrated at 0° C. to give 54 mg of crude, colorless oil. As the triflate is highly unstable, it was used directly without further purification.

### Racemic ethyl-1-(1,3-dioxoisoindolin-2-yl)-3-fluoro-4-(((trifluoromethyl)sulfonyl)oxy) cyclopentane-1-carboxylate. (3.30)

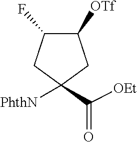

A scintillation vial under N₂ containing a stir bar, racemic ethyl-1-(1,3-dioxoisoindolin-2-yl)-3-fluoro-4-hydroxycyclopentane-1-carboxylate (3.29) (170 mg, 0.529 mmol, 1 equiv) and pyridine (94 μL, 1.17 mmol, 2.2 equiv) in DCM (2.5 mL) was cooled to 0° C. A separate vial containing trifluoromethanesulfonic anhydride (200 μL, 1.06 mmol, 2.0 equiv) in DCM (2.5 mL) was cooled to 0° C., and this mixture was added dropwise to the fluorohydrin solution with vigorous stirring. The mixture was stirred at 0° C. for 15 minutes, then diluted with hexanes (5 mL). A white powder precipitated and was filtered away, and the supernatant was concentrated in vacuo. The resulting crude residue was purified by silica gel flash chromatography, eluting the desired compound with a 15/85 EtOAc/hexanes gradient (R_{f}=0.2). White solid, 203 mg, 0.448 mmol, 85% yield. ¹H NMR (400 MHz, C₆D₆) δ 7.47-7.28 (m, 2H), 6.86-6.72 (m, 2H), 5.72-5.54 (m, 1H), 5.07-4.78 (m, 1H), 3.85-3.70 (m, 2H), 3.43 (dd, J=15.4, 8.4 Hz, 1H), 2.95 (ddd, J=21.6, 15.4, 6.6 Hz, 1H), 2.71 (ddd, J=20.5, 15.4, 5.1 Hz, 1H), 2.55 (dd, J=15.4, 5.8 Hz, 1H), 0.76 (t, J=7.1 Hz, 3H). ¹³C NMR (150 MHz, C₆D₆) δ 170.2, 168.2, 134.2, 131.8, 128.4, 123.3, 119.2 (q, J=319.7 Hz), 96.0 (d, J=185.3 Hz), 90.4 (d, J=29.4 Hz), 66.0 (d, J=3.1 Hz), 62.8, 39.6 (d, J=3.4 Hz), 39.4 (d, J=22.6 Hz), 13.7. ¹⁹F NMR (282 MHz, chloroform-d, fluorobenzene reference standard) δ −75.5, −181.9 (s, 3F), (dq, J=51.4, 20.2 Hz, 1F). IR (neat, cm⁻¹): 1774, 1736, 1720, 1709. HRMS (ESI) Calcd. for C₁₇H₁₆O₇NF₄S (M+H)⁺: 454.05781. Found: 454.05808. Melting point: 91-94° C.

Anti-Cis-3,4-[¹⁸F]-DFACPC ([¹⁸F]3.09)

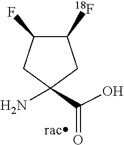 [SMILES: N[C@]1(C(=O)O)C[C@@H](F)[C@@H]([18F])C1]

To a glass vessel containing a solution of crypt-222 in MeCN (5.0 mg/mL) (1.0 mL) was added 790 mCi of no-carrier-added [¹⁸F]HF through a trap/release (T/R) cartridge by using a solution of K₂CO₃/H₂O (1.5 mg/mL) (0.6 mL). The solvent was removed at 110° C. with a nitrogen flow, and additional MeCN (3.5 mL) was added followed by evaporation of the solvent with a nitrogen flow to remove residual H₂O. Triflate precursor 3.19 (9 mg, 0.021 mmol) in dry MeCN (1 mL) was added to the vial, and the reaction mixture was heated at 110° C. for 10 min. The intermediate product was treated with 6 N HCl (0.5 mL) at 110° C. for 10 min and purified by passing through an IR column assembly consisting of a 7 mm×120 mm bed of AG 11A8 IR resin column, a neutral alumina SepPak™ (preconditioned with water), and an HLB Oasis™ reverse phase cartridge (preconditioned with water). [¹⁸F]3.09 eluted in series through the assembly with three successive portions of sterile saline (˜4.0 mL), into dose vials and was ready for in vitro and in vivo studies. Evidence of the identity of [¹⁸F]3.09 was achieved by comparing the R_{f} of the radioactive product visualized with radiometric TLC with the R_{f} of the authentic cold compound visualized with ninhydrin stain, using the solvent system MeCN/H₂O/CH₃OH=2:1:1 (R_{f}=0.6, Whatman silica gel plates). The only peak present on radiometric TLC analysis corresponded to [¹⁸F]3.09, and the radiochemical purity of the product exceeded 99%. The pH of the final dose solution was tested with pH paper and found to be 6-7. The isolated radiochemical yield was 50 mCi in 12 mL of saline as determined using a dose calibrator, affording a 10% decay corrected radiochemical yield based on a synthesis time of approximately 70 minutes, which proceeded immediately upon the end of cyclotron bombardment.

Racemic Trans-3,4-[¹⁸F]-DFACPC ([¹⁸F]3.23)

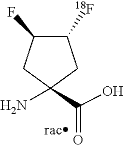 [SMILES: N[C@]1(C(=O)O)C[C@@H](F)[C@H]([18F])C1]

To a glass vessel containing a solution of Cryptand 222 in MeCN (22 mg/mL) (1.0 mL) was added 1460 mCi of no-carrier-added [¹⁸F]HF through a trap/release (T/R) cartridge by using a solution of Cs₂CO₃/H₂O (20 mg mg/mL) (0.6 mL). The solvent was removed at 110° C. with a nitrogen flow, and additional MeCN (3.5 mL) was added followed by evaporation of the solvent with a nitrogen flow to remove residual H₂O. Racemic triflate precursor 3.21 (20 mg, 0.047 mmol) in dry tBuOH (0.5 mL) and MeCN (0.5 mL) was added to the vial, and the reaction mixture was heated at 110° C. for 10 min. The intermediate product was treated with 6 N HCl (0.5 mL) at 110° C. for 10 min and purified by passing through an IR column assembly consisting of a 7 mm×120 mm bed of AG 11A8 IR resin, two neutral alumina SepPaks™ (preconditioned with water) and an HLB Oasis reverse phase cartridge (preconditioned with water). Racemic [¹⁸F]3.23 eluted in series through the assembly with three successive portions of sterile saline (˜4.0 mL), into dose vials and was ready for in vitro and in vivo studies. Based on analytical chiral HPLC data comparing the dose solution with authentic 3.23, [¹⁸F]3.23 was obtained in >99% radiochemical purity. The pH of the final dose solution was tested with pH paper and found to be 6-7. The isolated radiochemical yield was 12 mCi in 7 mL of saline as determined using a dose calibrator, affording a 1.3% decay corrected radiochemical yield based on a synthesis time of approximately 83 minutes, which proceeded immediately upon the end of cyclotron bombardment.

Racemic Syn-Cis-3,4-[¹⁸F]-DFACPC ([¹⁸F]3.33)

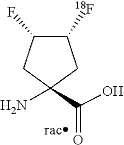 [SMILES: N[C@@]1(C(=O)O)C[C@@H]([18F])[C@@H](F)C1]

To a glass vessel containing a solution of Cryptand 222 in MeCN (22 mg/mL) (1.0 mL) was added 1460 mCi of no-carrier-added [¹⁸F]HF through a trap/release (T/R) cartridge by using a solution of Cs₂CO₃/H₂O (20 mg mg/mL) (0.6 mL). The solvent was removed at 110° C. with a nitrogen flow, and additional MeCN (3.5 mL) was added followed by evaporation of the solvent with a nitrogen flow to remove residual H₂O. Triflate precursor 3.30 (20 mg, 0.044 mmol) in dry tBuOH (0.5 mL) and MeCN (0.5 mL) was added to the vial, and the reaction mixture was heated at 110° C. for 10 min. The reaction mixture was diluted with 2 mL of acetonitrile, passed through two neutral alumina SepPaks™ (preconditioned with acetonitrile), and eluted with 10 mL of acetonitrile into a vented vial in a hot cell. The solvent was removed from the vial under a flow of nitrogen at 140° C. 0.5 mL of 2M NaOH was added to the vial which was sealed with a Teflon septum capped with an aluminum crimp top, and the mixture was heated to 140° C. for 5 minutes. The vial was cooled and 2 mL of concentrated HCl was added, and the sealed vial was again heated for 15 minutes at 140° C. Then vial was vented, and the solvent was removed under inert gas flow at 140° C. The resultant solid was taken up in H₂O (5 mL) and cannulated through a chain composed of one Waters HLB Oasis cartridge and two alumina SepPaks™ (both preconditioned with water). A second aliquot of water (5 mL) was passed through the chain, and the eluent containing [¹⁸F]3.33 was collected in a dose vial. The contents of the dose solution were assessed by analytical HPLC, utilizing [¹⁸F]3.23 as a reference. HPLC analysis indicated that [¹⁸F]3.33 was obtained in >99% radiochemical purity. The pH of the final dose solution was tested with pH paper and found to be 6-7. The isolated radiochemical yield was 8.4 mCi in 5 mL of water as determined using a dose calibrator, affording a 1.7% decay corrected radiochemical yield based on a synthesis time of approximately 135 minutes, which proceeded 35 minutes after the end of cyclotron bombardment (total time from end of bombardment to measurement of radiochemical yield was 170 minutes).

¹⁸F-Radiolabeled Trans Enantiomers of 3,4-DFACPC ([¹⁸F]3.23), Transport Properties in Cancer Cells, Biodistribution in Normal and Intracranial 9 L Gliosarcoma Bearing Fischer Rats

[¹⁸F]trans-3,4-DFACPC ([¹⁸F]3.23) was prepared in 1.3% decay corrected radiochemical yield in greater than 99% radio-chemical purity. Competitive uptake inhibition studies with 9 L gliosarcoma, human U87 ΔEGFR glioblastoma, and human DU145 androgen-independent prostate carcinoma cells to establish the mechanism of transport of [¹⁸F]3.23, as well as its affinity for each cell line. The biodistribution of [¹⁸F]3.23 was assessed in normal and intracranial 9 L gliosarcoma bearing rats via micro-PET imaging.

In each of the cell lines tested, [¹⁸F]3.23 was transported primarily via system L with some transport occurring via system ASC. In 9 L gliosarcoma and human U87 ΔEGFR glioblastoma cells, [¹⁸F]3.23 displayed uptake similar to the cis stereoisomers of 3,4-DFACPC and anti-1-amino-3-¹⁸F-fluorocyclobutane-1-carboxylic acid ([¹⁸F]-FACBC), an FDA approved PET tracer. In DU145 androgen-independent prostate carcinoma cells, [¹⁸F]3.23 displayed uptake 1.7 fold higher than [¹⁸F]-FACBC and nearly 2 fold higher than the best cis-3,4-DFACPC analog. MicroPET biodistribution studies in normal Fischer rats established that bladder accumulation of [¹⁸F]3.23 is delayed until at least 10 minutes post injection. In rats bearing intracranial 9 L gliosarcoma, [¹⁸F]3.23 gave tumor uptake values of up to 1.2% injected dose per gram (% ID/g) and tumor to contralateral brain tissue ratios of up to 2.8.

The delayed bladder accumulation of [¹⁸F]3.23 is sufficient to permit the imaging of tumors in the pelvic region, and the high affinity of [¹⁸F]3.23 for DU145 cells suggests that it is a highly promising preclinical candidate for imaging prostate cancer. Additionally, [¹⁸F]3.23 provided high tumor to normal brain tissue ratios in the 9 L gliosarcoma Fischer rat model, indicating that it may be useful for imaging intracranial tumors.

What is claimed is:

1. A composition comprising a compound having the following formula (I), 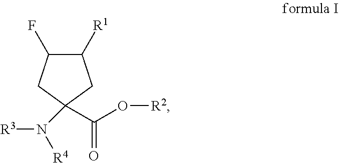
or salt thereof, wherein
R^{t} is a radionuclide, alkyl, halogen, hydroxy, amino, ((perfluoroalkyl)sulfonyl))oxy, (arylsulfonyl)oxy, mercapto, alkoxy, or alkylthio, or wherein R¹ is optionally substituted with one or more, the same or different, R¹⁰;
R² is hydrogen, alkyl, formyl, alkanoyl, benzoyl, carbocyclyl, aryl, or heterocyclyl, wherein R² is optionally substituted with one or more, the same or different, R¹⁰;
R³ is hydrogen, alkyl, cyano, hydroxy, amino, formyl, alkanoyl, benzoyl, carboxy, carbamoyl, alkylamino, (alkyl)₂amino, carbocyclyl, aryl, or heterocyclyl, wherein R³ is optionally substituted with one or more, the same or different, R¹⁰;
R⁴ is hydrogen or alkyl; or R³ and R⁴ and attached atoms come together to form a heterocyclyl optionally substituted with one or more, the same or different, R¹⁰;
R¹⁰ is alkyl, halogen, nitro, cyano, hydroxy, amino, mercapto, formyl, carboxy, carbamoyl, alkoxy, alkylthio, alkylamino, (alkyl)₂amino, alkylsulfinyl, alkyl sulfonyl, aryl sulfonyl, alkanoyl, benzoyl, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, propyl, tert-butyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethyl sulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, and heterocyclyl.

2. The composition of claim 1, wherein R¹ is a ¹⁸F.

3. The composition of claim 2, wherein R¹ is (trifluoromethyl)sulfonyl)oxy.

4. The composition of claim 1, wherein R² is ethyl.

5. The composition of claim 1, wherein R³ is tert-butoxycarbonyl and R⁴ is hydrogen.

6. The composition of claim 1, wherein R³ and R⁴ and attached atoms come together to form 1,3-dioxoisoindolin-2-yl.

7. The composition of claim 1 wherein the compound is 1-amino-3-fluoro-4-[¹⁸F]fluorocyclopentane-1-carboxylic acid or salt thereof.

8. The composition of claim 1 comprising a mixture of (1S,3R,4S)-1-amino-3-fluoro-4-[¹⁸F]fluorocyclopentane-1-carboxylic acid and (1R,3S,4R)-1-amino-3-fluoro-4-[¹⁸F]fluorocyclopentane-1-carboxylic acid (anti-cis-3,4-[¹⁸F]-DFACPC) or salt thereof.

9. The composition of claim 1 comprising a mixture of (1S,3S,4R)-1-amino-3-fluoro-4-[¹⁸F]fluorocyclopentane-1-carboxylic acid and (1R,3R,4S)-1-amino-3-fluoro-4-[¹⁸F]fluorocyclopentane-1-carboxylic acid (syn-cis-3,4-[¹⁸F]-DFACPC) of salt thereof.

10. The composition of claim 1 comprising a mixture of (1S,3R,4R)-1-amino-3-fluoro-4-[¹⁸F]fluorocyclopentane-1-carboxylic acid and (1R,3S,4S)-1-amino-3-fluoro-4-[¹⁸F]fluorocyclopentane-1-carboxylic acid (trans-3,4-[¹⁸F]-DFACPC) or salt thereof.

11. The composition of claim 1 wherein the compound ethyl 1-((tert-butoxycarbonyl)amino)-3-fluoro-4-(((trifluoromethyl)sulfonyl)oxy)cyclopentane-1-carboxylate or salt thereof.

12. The composition of claim 1 comprising a mixture of ethyl (1S,3R,4R)-1-((tert-butoxycarbonyl)amino)-3-fluoro-4-(((trifluoromethyl)sulfonyl)oxy)cyclopentane-1-carboxylate and ethyl (1R,3S,4S)-1-((tert-butoxycarbonyl)amino)-3-fluoro-4-(((trifluoromethyl)sulfonyl)oxy) cyclopentane-1-carboxylate or salts thereof.

13. The composition of claim 1 comprising a mixture of ethyl (1S,3R,4S)-1-((tert-butoxycarbonyl)amino)-3-fluoro-4-(((trifluoromethyl)sulfonyl)oxy)cyclopentane-1-carboxylate and ethyl (1R,3S,4R)-1-((tert-butoxycarbonyl)amino)-3-fluoro-4-(((trifluoromethyl) sulfonyl)oxy) cyclopentane-1-carboxylate or salts thereof.

14. The composition of claim 1 wherein the compound is ethyl 1-(1,3-dioxoisoindolin-2-yl)-3-fluoro-4-(((trifluoromethyl)sulfonyl)oxy)cyclopentane-1-carboxylate or salt thereof.

15. The composition of claim 1 comprising a mixture of ethyl (1S,3S,4S)-1-(1,3-dioxoisoindolin-2-yl)-3-fluoro-4-(((trifluoromethyl)sulfonyl)oxy)cyclopentane-1-carboxylate and ethyl (1R,3R,4R)-1-(1,3-dioxoisoindolin-2-yl)-3-fluoro-4-(((trifluoromethyl)sulfonyl)oxy)cyclo pentane-1-carboxylate or salts thereof.

16. A pharmaceutical composition comprising a compound of claim 1 or salts thereof and a pharmaceutically acceptable excipient.

17. The pharmaceutical composition of claim 16 in the form of a pH buffered aqueous salt solution between a pH of 4 and 6.

18. The pharmaceutical composition of claim 17 in the form of a saline citrate buffer or phosphate buffer, optionally comprising a saccharide or polysaccharide.

19. A method comprising:
a) administering a composition comprising a compound of claim 1 containing a radionuclide to a subject; and
b) scanning the subject for emissions.

20. The method of claim 19 further comprising the step of detecting the emissions and creating an image indicating or highlighting the location of the compound containing radionuclide in the subject.
